(19) United States
(12) Patent Application Publication
(10) Pub. No.: US 2011/0224147 A1
(43) Pub. Date: Sep. 15, 2011

(54) METHODS AND COMPOSITIONS

(75) Inventor: Colin Stanley Goodchild, Malvern (AU)

(73) Assignee: Relevare Aust. Pyt. Ltd., Abbotsford (AU)

(21) Appl. No.: 13/057,140

(22) PCT Filed: Jul. 31, 2009
(86) PCT No.: PCT/AU2009/000981

§ 371 Date: May 3, 2011

Related U.S. Application Data
(60) Provisional application No. 61/095,926, filed on Sep. 10, 2008.

(30) Foreign Application Priority Data
Aug. 1, 2008 (AU) 2008903953

(51) Int. Cl. A61K 38/17; A61K 31/497; A61K 31/675; A61K 31/535; A61K 38/08; A61K 38/12; A61K 38/06; A61P 25/00; A61P 25/04; A61P 25/02

(52) U.S. Cl. 514/18.1; 514/252.12; 514/79; 514/230.8; 514/21.8; 514/21.1; 514/21.9

(57) ABSTRACT

A method for inducing an analgesic response to inflammatory or neuropathic pain by administration of 1-(2-(4-chlorophenyl)-2-hydroxy)ethyl-4-(3,5-bis(1,1 dimethyl)-4-hydroxyphenyl)methyl piperazine, also called CNSB002 or AM-36, either alone or with an opioid and/or a neurokinin (NK) antagonist.

This application is associated with and claims priority from Australian Provisional Patent Application No. 2008903953, filed on 1 Aug. 2008 and U.S. Provisional Patent Application No. 61/095,296, filed on 10 Sep. 2008, the entire contents of which, are incorporated herein by reference.

### FIELD

The present invention relates generally to the field of pain management, and in particular, the management of inflammatory and neuropathic pain. More particularly, the present invention provides methods, protocols, compositions and devices which treat, alleviate, prevent, diminish or otherwise ameliorate the symptoms of pain.

### BACKGROUND

Bibliographic details of the publications referred to by author in this specification are collected alphabetically at the end of the description.

Reference to any prior art in this specification is not, and should not be taken as, an acknowledgment or any form of suggestion that this prior art forms part of the common general knowledge in any country.

Many pathological processes, in particular of a degenerative or traumatic nature, cause damage of peripheral nerves and CNS changes that persist after the acute disease process has settled (Backonja, Anesth Analg 97:785-790, 2003). These result in hypersensitivity and spontaneous firing of afferent nerve endings at the site of injury (Suzuki, Neuroreport 11 (12):R17-R21, 2000; Kajander and Bennett, J. Neurophysiol 68(3):734-744, 1992). Similar pathophysiological changes occur in the dorsal root ganglion cell bodies. This hyperexcitability is caused partly by an increased expression of sodium channels at the site of injury and also neurochemical remodelling in the spinal cord (Kajander et al, Neurosci Lett 138(2):225-228, 1992; Woolf, Nature 306:686-688, 1983; Chapman et al, Br J Pharmacol 116:1628-1634, 1995). These observations have led to the realization that it might be possible to develop drugs which selectively block these channels (Catterall, Trends Pharmacol Sci 8:57-65, 1987).

Many drugs in common clinical use for the treatment of pain states, including neuropathic pain, have as their mode of action blockade of sodium channels. These include: amitriptyline and other tricyclic antidepressants (Deffois et al, Neurosci Lett 220(2):117-120, 1996; Eisenberg et al, Neurology 57:505-509, 2001; Max et al, Neurology 37(4):589-596, 1987) anticonvulsants (MacDonald, Curr Opin Neurol 10:121-128, 1997); local anesthetic type (lidocaine) [Kastrup et al, Pain 28(1):69-75, 1987] and mexiletine Dejgard et al, Lancet 1 (8575-6):9-11, 1988; Chabal et al, Anesthesiology 76(4):513-517, 1992; Chou-Tan et al, Am J Phys Med Rehabil 75:84-87, 1996). However, effective doses may be difficult to achieve because of adverse effects. This is a common problem for the drugs used currently for treatment of persistent pain states. Often, the adverse side-effects of such remedies outweigh the benefits of treatment (Kalso et al, Eur J Pain 2(1):3-14, 1998; Chonjnowska, Anesth Analg 90:1007-1008, 2000; Tremont-Lukats et al, Anesth Analg 101:1738-1749, 2005).

There is a need to develop safe and efficacious therapies for inflammatory and neuropathic pain.

### SUMMARY

Methods and compositions for treating, alleviating, preventing, diminishing or otherwise ameliorating the symptoms associated with pain in a subject are provided. Reference to “pain” includes inflammatory and neuropathic pain as well as pain caused by disease conditions such as cancer. In particular, a method is contemplated for inducing an analgesic response to pain in a subject comprising the administration to the subject of an amount of CNSB002 either alone or in combination with an opioid such as morphine and/or an NK antagonist which administration is effective at reducing the level of or otherwise ameliorating the sensation of pain. In particular, CNSB002 on its own has reduced adverse side effects or when used in combination with an opioid and/or an NK antagonist, reduces the amount of opioid or NK antagonist required to be administered and hence adverse side effects.

CNSB002 is 1-(2-(4-chlorophenyl)-2-hydroxy)ethyl-4-(3,5-bis(1,1-dimethyl)-4-hydroxyphenyl)methylpiperazine (Jarrott et al, Drug Development Research 46:261-267, 1999). Reference to this chemical agent or its abbreviation “CNSB002” includes pharmaceutically acceptable salts.

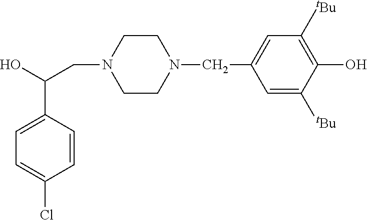

Accordingly, one aspect of the present invention contemplates a method for inducing an analgesic response to neuropathic or inflammatory pain in a subject comprising the administration of an amount of CNSB002 which is effective at reducing the level of or otherwise ameliorating the sensation of pain.

Another aspect of the present invention provides a method for inducing an analgesic response to pain in a subject comprising the administration of an amount of CNSB002 in combination with an opioid and/or an NK antagonist which is effective at reducing the level of or otherwise ameliorating the sensation of pain.

In an embodiment, the opioid is morphine. In another embodiment, the NK antagonist is any compound which inhibits, decreases, blocks or otherwise impairs the activity of substance P. Such compounds may either act directly by interacting with substance P or selectively interfere with any of the target receptors for substance P, such as NK1, NK2 or NK3 receptors. Hence, the present invention is further directed to the co-administration of CNSB002 and morphine and/or an NK antagonist to ameliorate the sensation of inflammatory, and neuropathic pain.

In another aspect, the present invention is directed to a method for inducing an analgesic response in a subject suffering from pain comprising the administration of CNSB002 concurrently, separately or sequentially with an opioid and/or an NK antagonist in combined amounts which is less than when any one of CNSB002, opioid or NK antagonist is used alone.

In a further aspect, the administration of CNSB002 alone or in combination with the opioid and/or NK antagonist does not cause overt sedation.

Opioids contemplated herein include morphine, fentanyl, oxycodone, codeine, dihydrocodeine, dihydrocodeinone enol acetate, desomorphine, apomorphine, diamorphine, pethidine, methadone, dextropropoxyphene, pentazocine, dextromoramide, oxymorphone, hydromorphone, dihydromorphine, papverine, papveretum, alfentanil, buprenorphine and tramadol and pharmaceutically acceptable salts, derivatives, homologs or analogs thereof.

Particular NK antagonists contemplated herein include NK1 antagonists. In a related aspect, the NK antagonist is aprepitant.

Yet another aspect relates to the use of CNSB002 either alone or in combination with an opioid and/or NK antagonist or pharmaceutically acceptable salts, derivatives, homologs or analogs thereof in the manufacture of a medicament for inducing an analgesic response in the treatment of pain. As indicated above, the pain is inflammatory or neuropathic pain.

Still another aspect is directed to the use of CNSB002 either alone or in combination with an opioid and/or an NK antagonist or pharmaceutically acceptable salts, derivatives, homologs or analogs thereof in the manufacture of a medicament for inducing an analgesic response in the treatment of neuropathic or inflammatory pain without inducing overt sedation.

A further aspect relates to the use of CNSB002 either alone or in combination with an opioid and/or an NK antagonist or pharmaceutically acceptable salts, derivatives, homologs or analogs thereof, in the manufacture of one or more separate or combined medicaments for inducing an analgesic response to pain. In an aspect, the analgesia is induced without overt sedation. In an embodiment the opioid is morphine.

In another embodiment, the NK antagonist is an NK1 antagonist.

Even yet another aspect is directed to the use of CNSB002 and one or more opioids and/or NK antagonists in the manufacture of a medicament for inducing analgesia in response to inflammatory pain. Still yet another aspect provides for the use of CNSB002 and one or more opioids and/or NK antagonists in the manufacture of a medicament for inducing analgesia in response to neuropathic pain.

In addition, the CNSB002 or CNSB002 and opioid or CNSB002 and NK antagonist or CNSB002, opioid and NK antagonist may be used in combination with one or more local anaesthetics such as but not limited to lignocaine, bupivacaine, ropivacaine, and procaine tetracaine or a pharmaceutically acceptable salt, derivative, homolog or analog thereof.

A delivery system is also provided for inducing analgesia in response to pain in a subject comprising CNSB002 and an opioid and/or an NK antagonist or a pharmaceutically acceptable salt, derivative, homolog or analog thereof. The delivery system may, for example, be in the form of a cream or an injection. The “injection” includes slow or controlled release injectables. The delivery system may also be a sustained release or slow release formulation, or a tamper proof formulation, or a pharmaceutical formulation or coated onto a stent, catheter or other mechanical device designed for use in a medical procedure.

The compounds according to the present invention may be administered, inter alia, orally, transmucosally, rectally including via suppository, subcutaneously, intravenously, intrathecally, epidurally, intramuscularly, intraperitoneally, intragastrically, intranasally, transdermally, transmucosally, including rectal, buccal (sublingual), transnasal administration or intestinally or injected into a joint.

Methods and compositions are provided herein for use in treating pain. In one embodiment, this occurs without causing overt sedation. As used herein, “without causing overt sedation” includes inducing an analgesic effect without causing significant cognitive or general impairment of nervous system function (such as attention or wakefulness). Such effects on cognition leads to a change in the measurement that leads to an erroneous conclusion about the drug combination causing analgesia.

The method of the present invention, in an embodiment, induces an analgesic response to pain without causing one or more dose-limiting side-effects. Dose-limiting side-effects include orthostatic hypotenstion, sinus bradycardia, neurocardiogenic syncope and hypotension.

In one aspect, CNSB002 is combined with an opioid and/or an NK antagonist or a pharmaceutically acceptable salt, derivative, homolog or analog thereof. The CNSB002, opioid and/or NK antagonist is administered, in one embodiment, at a dose of between about 0.1 mg/kg and about 50 mg/kg, at intervals of between about 1 hour and about 50 hours and may be administered prior to, simultaneously with each other.

In a particular embodiment, the subject is a mammal, and in a most particular embodiment, the subject is a human. The subject or a group of subjects may be selected on the basis of the type of pain experienced. The “type” of pain may also be subjectively determined based on symptoms described by the subject. Hence, a therapeutic protocol is contemplated which comprises selecting a subject on the basis of symptoms of pain and administering to the subject CNSB002 alone or CNSB002 and an opioid and/or an NK antagonist.

A further aspect provides a system for the controlled release of an active compound selected from CNSB002, CNSB002 and an opioid, CNSB002 and an NK antagonist and CNSB002 and an opioid and an NK antagonist, wherein the system comprises:

(a) a deposit-core comprising an effective amount of a first active compound and having defined geometric form, and

(b) a support-platform applied to the deposit-core, wherein the support-platform contains a second active compound, and at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

As used herein, the first active compound is one of (i) a CNSB002, an opioid or an NK antagonist; or (ii) the other of CNSB002, opioids or NK antagonist. The second active compound may be (i) or (ii) above.

In another aspect, a system is described for the controlled release for CNSB002 and an opioid and/or an NK antagonist wherein the system comprises:

(a) a deposit-core comprising an effective amount of the omega conotoxin and the neuronal excitation inhibitor; and

(b) a support platform applied to the deposit-core, the support platform comprising at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

Pain management protocols including point of care therapeutic protocols for controlling pain or the sensation of pain are also provided herein. The protocols include assessing a subject for pain type or causation of pain and providing to the subject CNSB002 alone or in combination with an opioid and/or an NK antagonist.

### BRIEF DESCRIPTION OF THE FIGURES

Some figures contain color representations or entities. Color photographs are available from the Patentee upon request or from an appropriate Patent Office. A fee may be imposed if obtained from a Patent Office

FIG. 1 is a diagrammatic representation of the protocol used in carrageenan-induced inflammatory pain.

FIG. 2 is a diagrammatic representation of the protocol used in streptozotocin (STZ)-induced diabetic neuropathy.

FIG. 3 is a graphical representation of the dose response curve for CNSB002 antinociception in carrageenan induced hyperalgesia: comparison with gabapentin and vehicle controls. Dose-response relationship for CNSB002 (0.5-10 mg/kg; ip)—antinociception in carrageenan-induced, paw inflammation in normal rats. Gabapentin (50 mg/kg; ip) and vehicle controls are also shown. Points shown are means and bars SEM. No dose of CNSB002 or gabapentin given alone caused significant antinociception in carrageenan paw inflammation (p>0.05; one way ANOVA with Tukey-Kramer post hoc test). However, the antinociception following 5 or 10 mg/kg CNSB002 was significantly greater than that caused by CNSB002 at the dose of 0.5 mg/kg.

FIG. 4 is a graphical representation of the dose response curve for morphine antinociception in carrageenan induced hyperalgesia: the effect of combined administration with CNSB002 5.0 mg/kg and comparison with vehicle controls. Dose-response relationships for morphine (0.2-3.2 mg/kg; ip) injected alone and co-administered with CNSB002 (5 mg/kg; ip) in normal rats—antinociception in carrageenan-induced paw inflammation. Vehicle controls and gabapentin (50 mg/kg co-administered with morphine 3.2 mg/kg) are also shown. Points shown are means and bars SEM.

No dose of morphine alone caused significant antinociception in the carrageenan paw inflammation model (p>0.05; one way ANOVA with Tukey-Kramer post hoc test). Morphine 3.2 mg/kg caused significant antinociception when co-administered with CNSB002 (5 mg/kg). No other dose of morphine caused significant antinociception in this model when co-administered either with 5 mg/kg CNSB002 or gabapentin 50 mg/kg (p<0.001; one way ANOVA with Tukey-Kramer post hoc). The antinociceptive effect of 3.2 mg/kg morphine and 5 mg/kg CNSB002 in combination was also greater than antinociception following administration of morphine alone at 0.2, 0.4 and 0.8 mg/kg (p<0.05; one way ANOVA with Tukey-Kramer post hoc test).

FIG. 5 is a graphical representation of the dose response curve for CNSB002 antinociception in hyperalgesia caused by STZ-induced diabetic neuropathy: comparison with gabapentin and vehicle controls. Dose-response relationship for CNSB002 (0.5-10 mg/kg; ip) in diabetic rats antinociceptive effect in reversal of hyperalgesia caused by streptozotocin-induced diabetic neuropathy. Gabapentin (50 mg/kg; ip) and vehicle controls are also shown. Points shown are means and bars SEM.

Both 10 mg/kg CNSB002 and 50 mg/kg gabapentin administered alone caused equally significant antinociception in diabetic neuropathy (p<0.01; one way ANOVA with Tukey-Kramer post hoc test). Antinociception following 10 mg/kg CNSB002 was significantly greater than that caused by CNSB002 at the doses of 0.5, 1.0 and 2.5 mg/kg, but not 5 mg/kg.

FIG. 6 is a graphical representative of the dose response curve for morphine antinociception in STZ-induced diabetic neuropathy: the effect of combined administration with CNSB002 5.0 mg/kg and comparison with gabapentin and vehicle controls. Dose-response relationships for morphine (0.2-3.2 mg/kg; ip) injected alone and co-administered with CNSB002 (5 mg/kg; ip) in diabetic rats: antinociception in reversal of hyperalgesia in streptozotocin-induced diabetic neuropathy. Vehicle controls and the combination of gabapentin (50 mg/kg; ip) with morphine (3.2 mg/kg; ip) are also shown. Points shown are means and bars SEM.

No dose of morphine alone caused significant antinociception in this model of diabetic neuropathic pain (p>0.05; one way ANOVA with Tukey-Kramer post hoc test) except for the highest dose (3.2 mg/kg; p<0.05—one way ANOVA with Tukey-Kramer post hoc test). By contrast dose combinations of morphine with 5 mg/kg CNSB002 containing more than 0.2 mg/kg morphine caused significant antinociception compared with vehicle controls (p<0.001; one way ANOVA with Tukey-Kramer post hoc test).

Gabapentin co-administered with morphine also caused significant antinociception compared with vehicle controls (p<0.001; one way ANOVA with Tukey-Kramer post hoc test), and this was not statistically significantly different from the antinociceptive effect of the same dose of morphine given in combination with CNSB002 (5 mg/kg; ip). the antinociception, following co-administration of 5 mg/kg CNSB002 with morphine at 3.2 and 0.8 mg/kg was significantly greater than morphine administered at 3.2 and 0.8 mg/kg alone (p<0.05; one way ANOVA with Tukey-Kramer post hoc test). By contrast the morphine/gabapentin combination (3.2/50 mg/kg; ip) did not cause significantly greater antinociception compared with morphine administered alone (3.2 mg/kg; ip).

### DETAILED DESCRIPTION

Throughout the specification and the claims which follow, unless the context requires otherwise, the word “comprise”, and variations such as “comprises” and “comprising” will be understood to imply the inclusion of a stated integer or step or group of integers or steps but not the exclusion of any other integer or step or group of integers or steps.

Reference to any prior art in this specification is not, and should not be taken as an acknowledgement or any form of suggestion that this prior art forms part of the common general knowledge in any country.

The singular forms “a”, “an” and “the” include plural aspects unless the context clearly dictates otherwise. Thus, for example, reference to “a pain” includes single or multiple types of pain; reference to “an opioid” or “an NK antagonist” includes a single opioid or NK antagonist, as well as two or more opioids or NK antagonists; reference to “the invention” includes one aspect or multiple aspects of the invention.

Terms such as “effective amount”, “therapeutically effective amount” and “an analgesic effective amount” of an agent as used herein mean a sufficient amount of the agent (e.g. CNSB002 alone or in combination with an opioid) and/or an NK antagonist to provide the desired therapeutic or physiological effect or outcome, which includes achievement of pain reduction such as a sense of analgesia. In particular, pain relief is in respect of inflammatory or neuropathic pain. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation or the experience of the clinician. In particular, the methods and compositions described herein including the therapeutic protocol achieve analgesia of pain. In an embodiment, analgesia is achieved without overt sedation. Effective amounts of each of CNSB002, opioid and NK antagonist is considered from about 0.1 mg/kg to 50 mg/kg body weight. In a particular embodiment, the amount used of any one component is less when measured in combination compared to what is required if the component is used alone.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, amideamide, prodrug or derivative of a compound is a salt, ester, amide, prodrug or derivative that this not biologically or otherwise undesirable.

The terms “treating” and “treatment” as used herein refer to reduction in severity and/or frequency of pain associated with the condition being treated, elimination of symptoms and/or underlying cause of the pain, prevention of the occurrence of pain associated with a condition and/or its underlying cause and improvement or remediation or amelioration of pain following a condition. Hence, the treatment proposed herein reduces pain but this may be independent of the condition being treated.

“Treating” a subject may involve both treating the condition and reducing pain. A prophylactic component of pain relief is also contemplated in anticipation of a treatment regime which might cause pain.

A “subject” as used herein refers to an animal, including a mammal such as a human who can benefit from the pharmaceutical formulations and methods of the present invention. There is no limitation on the type of animal that could benefit from the presently described pharmaceutical formulations and methods. A subject regardless of whether a human or non-human animal may be referred to as a subject, individual, patient, animal, host or recipient. The compounds and methods described herein have applications in human medicine, veterinary medicine as well as in general, domestic or wild animal husbandry.

The term “mammal” includes humans and non-human primates such as orangutangs, gorillas and marmosets as well as livestock animals, laboratory test animals, companion animals or captive wild animals, and avian species.

Examples of laboratory test animals include mice, rats, rabbits, simian animals, guinea pigs and hamsters. Rabbits, rodent and simian animals provide a convenient test system or animal model. Livestock animals include sheep, cows, pigs, goats, horses and donkeys.

In one aspect, a method is provided for inducing an analgesic response to pain in a subject. The pain is generally inflammatory or neuropathic pain. As indicated above, the term “subject” is intended to include and encompass both humans and other mammals. This aspect also includes, in one embodiment, the step of selecting a subject having inflammatory or neuropathic pain to be a recipient of treatment. The selection process includes an assessment of symptoms of pain or symptoms of a condition likely to result in pain.

The term “pain” is intended to describe the subset of acute and chronic pain that results from inflammatory or neuropathic pain. Pain from considerations such as cancer conditions is also contemplated.

Throughout this specification, the term “neuropathic pain” is to be understood to mean pain initiated or caused by a primary lesion or dysfunction within the nervous system. Examples of categories of neuropathic pain that may be treated by the methods of the present invention include monoradiculopathies, trigeminal neuralgia, postherpetic neuralgia, phantom limb pain, complex regional pain syndromes, back pain, neuropathic pain associated with AIDS and infection with the human immunodeficiency virus and the various peripheral neuropathies, including, but not limited to drug-induced and diabetic neuropathies. Neuropathic pain is a distinct entity from nociceptive pain which is caused by activation of nociceptors and includes pain caused by cuts, bruises, bone fractures, crush injuries, burns, or tissue trauma.

In a further embodiment, the present invention extends to treating pain associated with any one or more of the following diseases which cause neuropathic pain or which have a neuropathic pain component: abdominal wall defect, abdominal migraine, achondrogenesis, achondrogenesis Type IV, achondrogenesis Type III, achondroplasia, achondroplasia tarda, achondroplastic dwarfism, acquired humanimmunodeficiency syndrome (AIDS), acute intermittent porphyria, acute porphyrias, acute shoulder neuritis, acute toxic epidermolysis, adiposa dolorosa, adrenal neoplasm, adrenomyeloneuropathy, adult dermatomyositis, amyotrophic lateral sclerosis, amyotrophic lateral sclerosis-polyglucosan bodies, AN, AN 1, AN 2, anal rectal malformations, anal stenosis, arachnitis, arachnoiditis ossificans, arachnoiditis, arteritis giant cell, arthritis, arthritis urethritica, ascending paralysis, astrocytoma grade I (Benign), astrocytoma grade II (Benign), athetoid cerebral palsy, Barrett esophagus, Barrett ulcer, benign tumors of the central nervous system, bone tumor-epidermoid cyst-polyposis, brachial neuritis, brachial neuritis syndrome, brachial plexus neuritis, brachial-plexus-neuropathy, brachiocephalic ischemia, brain tumors, brain tumors benign, brain tumors malignant, brittle bone disease, bullosa hereditaria, bullous cie, bullous congenital ichthyosiform erythroderma, bullous ichthyosis, bullous pemphigoid, Burkitt's lymphoma, Burkitt's lymphoma African type, Burkitt's lymphoma non-African type, calcaneal valgus, calcaneovalgus, cavernous lymphangioma, cavernous malformations, central form neurofibromatosis, cervical spinal stenosis, cervical vertebral fusion, Charcot's disease, Charcot-Marie-Tooth disease, Charcot-Marie-Tooth disease variant, Charcot-Marie-Tooth-Roussy-Levy disease, childhood dermatomyositis, chondrodysplasia punctata, chondrodystrophia calcificans congenita, chondrodystrophia fetalis, chondrodystrophic myotonia, chondrodystrophy, chondrodystrophy with clubfeet, chondrodystrophy epiphyseal, chondrodystrophy hyperplastic form, chondroectodermal dysplasias, chondrogenesis imperfecta, chondrohystrophia, chondroosteodystrophy, chronic adhesive arachnoiditis, chronic idiopathic polyneuritis (CIP), chronic inflammatory demyelinating polyneuropathy, chronic inflammatory demyelinating polyradiculoneuropathy, cicatricial pemphigoid, complex regional pain syndrome, congenital cervical synostosis, congenital dysmyelinating neuropathy, congenital hypomyelinating polyneuropathy, congenital hypomyelination neuropathy, congenital hypomyelination, congenital hypomyelination (onion bulb) polyneuropathy, congenital ichthyosiform erythroderma, congenital tethered cervical spinal cord syndrome, cranial arteritis, Crohn's disease, cutaneous porphyrias, degenerative lumbar spinal stenosis, demyelinating disease, diabetes mellitus diabetes insulin dependent, diabetes mellitus, diabetes mellitus Addison's disease myxedema, discoid lupus, discoid lupus erythematosus, disseminated lupus erythematosus, disseminated neurodermatitis, disseminated sclerosis, eds kyphoscoliotic, eds kyphoscoliosis, eds mitis type, eds ocular-scoliotic, elastosis dystrophica syndrome, encephalofacial angiomatosis, encephalotrigeminal angiomatosis, enchondromatosis with multiple cavernous hemangiomas, endemic polyneuritis, endometriosis, eosinophilic fasciitis, epidermolysis bullosa, epidermolysis bullosa acquisita, epidermolysis bullosa hereditaria, epidermolysis bullosa letalias, epidermolysis hereditaria tarda, epidermolytic hyperkeratosis, epidermolytic hyperkeratosis, familial lumbar stenosis, familial lymphedema praecox, fibromyalgia, fibromyalgia-fibromyositis, fibromyositis, fibrositis, fibrous ankylosis of multiple joints, fibrous dysplasia, fragile x syndrome, generalized fibromatosis, guillain-bane syndrome, hemangiomatosis chondrodystrophica, hereditary sensory and autonomic neuropathy type I, hereditary sensory and autonomic neuropathy type II, hereditary sensory and autonomic neuropathy type III, hereditary sensory motor neuropathy, hereditary sensory neuropathy type I hereditary sensory neuropathy type i, hereditary sensory neuropathy type II, hereditary sensory neuropathy type M, hereditary sensory radicular neuropathy type I, hereditary sensory radicular neuropathy type I, hereditary sensory radicular neuropathy type II, herpes zoster, Hodgkin disease, Hodgkin's disease, Hodgkin's lymphoma, hyperplastic epidermolysis bullosa, hypertrophic interstitial neuropathy, hypertrophic interstitial neuritis, hypertrophic interstitial radiculoneuropathy, hypertrophic neuropathy of refsum, idiopathic brachial plexus neuropathy, idiopathic cervical dystonia, juvenile (childhood) dermatomyositis (jdms), juvenile diabetes, juvenile rheumatoid arthritis, pes planus, leg ulcer, lumbar canal stenosis, lumbar spinal stenosis, lumbosacral spinal stenosis, lupus, lupus, lupus erythematosus, lymphangiomas, mononeuritis multiplex, mononeuritis peripheral, mononeuropathy peripheral, monostotic fibrous dysplasia, multiple cartilaginous enchondroses, multiple cartilaginous exostoses, multiple enchondromatosis, multiple myeloma, multiple neuritis of the shoulder girdle, multiple osteochondromatosis, multiple peripheral neuritis, multiple sclerosis, musculoskeletal pain syndrome, neuropathic amyloidosis, neuropathic beriberi, neuropathy of brachialpelxus syndrome, neuropathy hereditary sensory type I, neuropathy hereditary sensory type II, Nieman pick disease type a (acute neuronopathic form), Nieman pick disease type b, Nieman pick disease type c (chronic neuronopathic form), non-scarring epidermolysis bullosa, ochronotic arthritis, ocular herpes, onion-bulb neuropathy, osteogenesis imperfect, osteogenesis imperfecta, osteogenesis imperfecta congenita, osteogenesis imperfecta tarda, peripheral neuritis, peripheral neuropathy, perthes disease, polyarteritis nodosa, polymyalgia rheumatica, polymyositis and dermatomyositis, polyneuritis peripheral, polyneuropathy peripheral, polyneuropathy and polyradiculoneuropathy, polyostotic fibrous dysplasia, polyostotic sclerosing histiocytosis, postmyelographic arachnoiditis, primary progressive multiple sclerosis, psoriasis, radial nerve palsy, radicular neuropathy sensory, radicular neuropathy sensory recessive, reflex sympathetic dystrophy syndrome, relapsing-remitting multiple sclerosis, sensory neuropathy hereditary type I, sensory neuropathy hereditary type II, sensory neuropathy hereditary type I, sensory radicular neuropathy, sensory radicular neuropathy recessive, sickle cell anemia, sickle cell disease, sickle cell-hemoglobin c disease, sickle cell-hemoglobin d disease, sickle cell-thalassemia disease, sickle cell trait, spina bifida, spina bifida aperta, spinal arachnoiditis, spinal arteriovenous malformation, spinal ossifying arachnoiditis, spinal stenosis, stenosis of the lumbar vertebral canal, still's disease, syringomyelia, systemic sclerosis, talipes calcaneus, talipes equinovarus, talipes equinus, talipes varus, talipes valgus, tandem spinal stenosis, temporal arteritis/giant cell arteritis, temporal arteritis, tethered spinal cord syndrome, tethered cord malformation sequence, tethered cord syndrome, tethered cervical spinal cord syndrome, thalamic pain syndrome, thalamic hyperesthetic anesthesia, trigeminal neuralgia, variegate porphyria, vertebral ankylosing hyperostosis amongst others.

The term “inflammatory pain” or a pain associated with inflammation is intended to describe the subset of acute and chronic pain that results from inflammatory processes, such as may arise in the case of infections, arthritis and neoplasia or tumor related hypertrophy. Inflammatory pain includes pain associated with rheumatoid arthritis, osteo-arthritis, psoriatic arthropathy, arthritis associated with other inflammatory and autoimmune conditions, degenerative conditions such as back strain and mechanical back pain or disc disease, post operative pain, pain from an injury such as a soft tissue bruise or strained ligament or broken bone, abscess or cellulitis, fibrositis or myositis.

In another embodiment, the present invention contemplates the use of compositions and methods comprising CNSB002 either alone or in combination with an opioid and/or an NK antagonist in the treatment of pain associated with inflammatory conditions. Examples of inflammatory conditions include, but are not limited to, inflammatory diseases and disorders which result in a response of redness, swelling, pain, and a feeling of heat in certain areas that is meant to protect tissues affected by injury or disease. Inflammatory diseases which include a pain component which can be relieved using the compositions and methods of the present invention include, without being limited to, acne, angina, arthritis, aspiration pneumonia, disease, empyema, gastroenteritis, inflammation, intestinal flu, NEC, necrotizing enterocolitis, pelvic inflammatory disease, pharyngitis, PID, pleurisy, raw throat, redness, rubor, sore throat, stomach flu and urinary tract infections, chronic inflammatory demyelinating polyneuropathy, chronic inflammatory demyelinating Polyradiculoneuropathy, chronic inflammatory demyelinating polyneuropathy, chronic inflammatory demyelinating polyradiculoneuropathy.

In a further embodiment, the present invention provides methods and compositions for alleviating the pain associated with cancer.

In one particular embodiment, CNSB002, either alone or in combination with an opioid and/or an NK antagonist is used during or following cancer treatment. Examples of cancers which contain a pain component which may be relieved using the compositions and methods of the present invention include but are not limited to abll protooncogene, aids related cancers, acoustic neuroma, acute lymphocytic leukaemia, acute myeloid leukaemia, adenocystic carcinoma, adrenocortical cancer, agnogenic myeloid metaplasia, alopecia, alveolar soft-part sarcoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumors, breast cancer, CNS tumors, carcinoid tumors, cervical cancer, childhood brain tumors, childhood cancer, childhood leukaemia, childhood soft tissue sarcoma, chondrosarcoma, choriocarcinoma, chronic lymphocytic leukaemia, chronic myeloid leukaemia, colorectal cancers, cutaneous T-cell lymphoma, dermatofibrosarcoma-protuberans, desmoplastic-small-round-cell-tumor, ductal carcinoma, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibrosarcoma, gall bladder cancer, gastric cancer, gastrointestinal cancers, gastrointestinal-carcinoid-tumor, genitourinary cancers, germ cell tumors, gestational-trophoblastic-disease, glioma, gynaecological cancers, haematological malignancies, hairy cell leukaemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhan's-cell-histiocytosis, laryngeal cancer, leiomyosarcoma, leukaemia, Li-fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid-tumor-of-kidney, medulloblastoma, melanoma, merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, nijmegen breakage syndrome, non-melanoma skin cancer, non-small-cell-lung-cancer-(NSCLC), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumors, pituitary cancer, polycythemia vera, prostate cancer, rare-cancers- and -associated-disorders, renal cell carcinoma, retinoblastoma, rhabdomyosarcoma, Rothmund-Thomson syndrome, salivary gland cancer, sarcoma, schwannoma, Sezary syndrome, skin cancer, small cell lung cancer (SCLC), small intestine cancer, soft tissue sarcoma, spinal cord tumors, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvi-/-ureter), trophoblastic cancer, urethral cancer, urinary system cancer, uroplakins, uterine sarcoma, uterus cancer, vaginal cancer, vulva cancer, Waldenstrom's-macroglobulinemia or Wilms' tumor.

In an embodiment, an analgesic response is induced without inducing overt sedation to pain being suffered by a subject, including a human subject. A subject, in this context, may also be referred to as a “patient”, “target”, “recipient” or “individual”. In this context the terms “analgesia” and “analgesic response” are intended to describe a state of reduced sensibility to pain, which occurs without overt sedation and in an embodiment without an effect upon the sense of touch. In another aspect, the sensibility to pain is completely, or substantially completely, removed. To assess the level of reduction of sensibility to pain associated with the analgesia induced by the methods according to the present invention it is possible to conduct tests such as the short form McGill pain questionnaire and/or visual analog scales for pain intensity and/or verbal rating scales for pain intensity and/or measurement of tactile allodynia using von Frey hairs or similar device. These tests are standard tests within the art and would be well known to the skilled person. Hence, a reduction to the sensibility to pain can be represented subjectively or qualitatively as a percentage reduction by at least 10%, at least 20%, at least 50%, at least 70% or at least 85% including at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54; 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or 85%.

Accordingly, a method is contemplated for inducing an analgesic response to inflammatory or neuropathic pain in a subject comprising the administration to the subject an amount of CNSB002, or a pharmaceutically acceptable salt thereof, effective to reduce the level of or otherwise ameliorate the sensation of pain. In another embodiment, a method is provided for inducing an analgesic response to pain comprising the administration to the subject an amount of CNSB002 and an opioid or pharmaceutically acceptable salts, derivatives, homologs or analogs thereof, effective to reduce the level of or otherwise ameliorate the sensation of pain. In another embodiment, a method is provided for inducing an analgesic response to pain comprising the administration to the subject an amount of CNSB002 and an NK antagonist and an opioid or pharmaceutically acceptable salts, derivatives, homologs or analogs thereof, effective to reduce the level of or otherwise ameliorate the sensation of pain. In another embodiment, a method is provided for inducing an analgesic response to pain comprising the administration to the subject an amount of CNSB002, opioid and an NK antagonist and an opioid or pharmaceutically acceptable salts, derivatives, homologs or analogs thereof, effective to reduce the level of or otherwise ameliorate the sensation of pain. Reference to “CNSB002” means 1-(2-(4-chlorophenyl)-2-hydroxy)ethyl-4-(3,5-bis(1,1-dimethyl)-4-hydroxyphenyl)methyl-piperazine as well as its pharmaceutically acceptable salts thereof. Also contemplated by the present invention are derivatives of CNSB002, such as those described in International Patent Application No. PCT/AU97/00293 (WO 97/43259).

In a related aspect, the present invention provides a method for inducing an analgesic response to pain in a subject comprising the administration to the subject an amount of CNSB002 in combination with an opioid and/or an NK antagonist effective to reduce the level of or otherwise ameliorate the sensation of pain.

In another aspect, the present invention provides a method for inducing an analgesic response without overt sedation to pain in a subject comprising the administration to the subject an amount of CNSB002 in combination with an opioid and/or an NK antagonist effective to reduce the level of or otherwise ameliorate the sensation of pain.

Another aspect provides a method of inducing analgesia in a subject suffering pain by administering to the subject CNSB002 concurrently, separately or sequentially with respect to an opioid and/or an NK antagonist or a pharmaceutically acceptable salt, derivative, homolog or analog thereof, in an amount effective to reduce the level of or otherwise ameliorate the sensation of pain associated with inflammatory or neuropathic pain.

Still another aspect contemplates combination therapy in the treatment of pain wherein the treatment of the disease, condition or pathology is conducted in association with pain management using CNSB002 and an opioid and/or an NK antagonist or a pharmaceutically acceptable salt, derivative, homolog or analog thereof and optionally in addition to an analgesic agent.

In yet another aspect contemplates combination therapy in the treatment of pain without inducing overt sedation wherein the treatment of the disease, condition or pathology is conducted in association with pain management using CNSB002 and an opioid and/or an NK antagonist or a pharmaceutically acceptable salt, derivative, homolog or analog thereof and optionally in addition to an analgesic agent.

Yet another aspect is directed to a method for inducing an analgesic response to pain in a subject comprising administering to the subject an amount of CNSB002 alone or in combination with an opioid and/or an NK antagonist and a local anaesthetic such as lignocaine, bupivacaine, ropivacaine, and procaine tetracaine or a pharmaceutically acceptable salt, derivative, homolog or analog thereof to reduce the level of or otherwise ameliorate the sensation of pain.

Combinations of pain relief components contemplated herein, therefore, include CNSB002, CNSB002 and an opioid, CNSB002 and an NK antagonist and CNSB002, an opioid and an NK antagonist.

By the term “overt sedation” it is intended to convey that the methods (and compositions) described herein do not result in a level of sedation of the patient or subject being treated which shows significant, visible or apparent drowsiness or cause unconsciousness of the patient being treated. Thus, the treatment methods and compositions herein, in one embodiment, do not result in sleepiness or drowsiness in the patient that interfere with, or inhibit, the activities associated with day to day living, such as driving a motor vehicle or operating machinery for human subjects, or feeding and grooming for animal subjects. The term “without overt sedation” also means inducing an analgesic effect without causing significant cognitive or general impairment of nervous system function (such as attentiveness or wakefulness). Such effects on cognition can lead to a change in the measurement that leads to an erroneous conclusion about the level or type of pain or effect of amelioration of symptoms.

As used herein, opioid compounds (opioids) include any compound that is physiologically acceptable in animal systems and is a full or at least partial agonist of an opioid receptor. Opioid compounds are well known and include naturally occurring compounds derived from opium such as morphine, codeine, and papavarine as well as derivatives of such compounds that generally have structural similarity as well as other structurally unrelated compounds that agonise an opioid receptor present in a mammalian system. Specific examples of opioid compounds contemplated by the present invention include: morphine, fentanyl, oxycodone, codeine, dihydrocodeine, dihydrocodeinone enol acetate, desomorphine, apomorphine, diamorphine, pethidine, methadone, dextropropoxyphene, pentazocine, dextromoramide, oxymorphone, hydromorphone, dihydromorphine, nalbuphine papaverine, papaveretum, alfentanil, buprenorphine and tramadol and pharmaceutically acceptable salts, derivatives, homologs or analogs thereof.

The term “NK antagonist” is intended to encompass known and as yet unknown compounds (including pharmaceutically acceptable salts, derivatives, homologs or analogs thereof) which inhibit, decrease or block, or otherwise impair the activity of neurokinin 1, neurokinin 2 or substance P. Such compounds can act directly on neurokinin 1, neurokinin 2 or substance P to inhibit its activity or can act on the family of NK receptors such as NK1, NK2 or NK3 receptors. Examples of such agents include achiral pyridine class of neurokinin-1 receptor antagonists; aprepitant; netupitant 21; betctupitant 29; elzlopitant; lanepitant; osanetant; talnetant; GR205171; MK 0517; MK517; MEN 11467; nepadutant; MEN 11420; M274773; [Sar (9), Met (02) (11)]-Substance P; Tyr (6), D-Phe (7), D-His (9)-Substance-P (6-11) (sendide); (beta;-Ala(8))-Neurokinin A (4-10); (Tyr(5), D-Trp (6,8,9), Lys-NH(2) (10))-Neurokinin A; [D-Proz, D-Trip 7,9]-SP DPDT-SP; [D-Proz, D-Phe7, D-Trp9]-SP; SR48968 and 4-Alkylpiperidine derivative; telnetant; SB223412; SB223412A; telnetant hydrochloride; MDL103392; phosphorylated morpholine acetal human neurokinin-1 receptor agonists; SDZ NKT 343; LY 303 870; Ym-35375 and spiro-substituted piperidines; YM-44778; YM-38336; Septide; L732,13; Dactinomyan analogues; MEN 10207; L 659874; L 668,169; FR113680 and derivative; GR 83074; tripeptides possersi, the glutaminyl-D-trypto phy phenyl alonite sequence; L 659,877; R396; Imidazo[4,5-b]quinoxaline cyonines as neurokinin antagonists; MEN 10208; DPDTP-octa; GR73632; GR64349; senktide; GR71251; [D-Arg1, D-Pro2, D-Trp 7,9, Leu11]-SP (1-11); Ac heu-Asp-Gln-Trp-Phe-Gly NH2; Thr-Asp-Tyr-D-Tvp-Val-D-Trp-D-Trp-Arg NH2; Cyclo [Eln-Trp-Phe-Gly-Leu-Met]; D-Pro2D-Trp 7,9; D-Arg1D-Trp 7,9 leu11; [Gly6]-NKB [3-10]; [Arg3, D-Ala6]-NKB [3-10]; CP-9634; 3 aminoquinudidine; CP-99994; S18525; S19752; 4-quinoline carboxinide fremincik class; CP-122721; MK-869; GR205171; Spantide II; CP-96,345; L703,606; SR140, DNK333; 2-phenyl-4-quinolinecarboximides class; FK224; FR 115224; FK888; ZM253270-yrrolopyrimidine class of nonpeptide neurokinin antagonists; GR71251; GR82334; RP67580; diacylpiperazine antagonists of human neurokinin eg L-161664; RP67580; MEN10376; GR98400; N2-[N2-(1H-indol-3-ylcarbonyl)-L-lysyl]-N-methyl-N-(phenyl-methyl)-L-phenylalaninamibe (2b); SP-(1-11); SP-(6-11); SP-(4-11) WIN51703; Spantide II; Spantide III; Spantide I; L754030; MK0869; ONO-7436; ONO 7436; MEN13510; 1-[2-(R)-{1-1R)-[3,5-bis(trifluoromethyl)phenyl]ethoxy}-3-(R)-(3,4-difluorophenyl)-4-(R)-tetrahydro-2H-pyran-4-ylmethyl]-3-(r)-methylpiperidine-3-carboxylic acid (1); LY 306,740; SLV-323; 2-substituted-4-aryl-6,7,8,9-tetrahydro-5H-pyrimido[4,5-b][1,5]oxazocin-5-one; 9-substituted-7-aryl-3,4,5,6-tetrahydro-2H-pyrido[4,3-b]- and [2,3-b]-1,5-oxazocin-6-one; SR142801; SB222200; CP96345; SR48968; ezlopitant; CJ 11974; MEN11558; [18F] SPA-RQ; neuropitant 21; betupitant 29; SR 144190; SR48692; SR141716; L733060; vofopitant; R-673; nepadutant; saredutant; UK 290795; 2-(4-biphenylyl)quinoline-4-carboxylate and carboxamide analogs (neurokinin-3 receptor antagonist); 4-Amino-2-(aryl)-butylbenzamides and analogues; MK-869; L742694; CP 122721; 1-alkyl-5-(3,4-dichlorophenyl)-5-[2-[(3-substituted)-1-azetidinyl]ethyl]-2-piperidines; L760735; L758,298, Cbz-Gly-Leu-Trp-0Bzl(CF(3))(2); L733,061; SR144190; SB235375; N—[(R,R)-(E)-1-arylmethyl-3-(2-oxo-azepan-3-yl) carbamoyl]allyl-N-methyl-3,5-bis(trifluoromethyl)benzamides; 3-[N¹-3,5-bis(trifluoromethyl)benzoyl-N-arylmethyl-N¹-methylhydrazino]-N—[(R)-2-oxo-azepan-3-yl]propionanides; SR142806; SR48,968; CP141,938; LY306740; SB40023; SB414240; Nolpitantium; SR140333; perhydroisoindole RP 67580, Depitant; RPR 100893; Lanepitant; LY-303870; LY303870; sanoti synthelabo; nolpitanium; SR 140333; SR 48968; Savedutant; AV 608; AV-608, AV608; CGP 60829; NK-608; NKP-608C; NKP608; CS003; R113281; Vestipitant; 597599; GW 597599; GW 597599B; Nurokinin antagonist; SSR 240600; casopitant; 679769; GW 679769; TA 5538; SSR 146977; SLV317; SLV-317; 823296; GW 823296; AVE 5883; AVE-5883; AZ 311; SB 235375; SB 733210; AZ 685; SAR 102279; SAR 10279; SSR 241586; SLV 332; Neurokinin 2 antagonist-Solvay; NK-2 antagonist-Solvat; SLV-332; SLV332, NIK 616; MPV4505; NIK616; MPC 4505; Z501; Z-501; 1 TAK 637; CP 96345; L 659877; CGP 49823; GR 203040; L 732138; S 16474; WIN 51708; ZD 7944; S 18523; CI 1021; PD 154075; 758298; ZD 4974; S 18920; HMR 2091; FK 355; SCH 205528; NK 5807; NIP 531; SCH 62373; UK 224671; MEN 10627; WIN 64821; MDL 105212A; MEN 10573; TAC 363; 1 MEN 11149; HSP 117; NIP 530; and AZD 5106.

The phrase “pharmaceutically acceptable salt or derivative” is intended to convey any pharmaceutically acceptable tautomer, salt, pro-drug, hydrate, solvate, metabolite or other compound which, upon administration to the subject, is capable of providing (directly or indirectly) the compound concerned or a physiologically (e.g. analgesically) active compound, metabolite or residue thereof. An example of a suitable derivative is an ester formed from reaction of an OH or SH group with a suitable carboxylic acid, for example C₁₋₃alkyl-CO₂H, and HO₂C—(CH₂)ₙ—CO₂H (where n is 1-10 such as 1, 2, 3, 4, 5, 6, 7, 8, 9, 10, but particularly 1-4), and CO₂H—CH₂phenyl.

Thus, the active compounds may be in crystalline form, either as the free compounds or as solvates (e.g. hydrates). Methods of solvation are generally known within the art.

The salts of the active compounds of the invention are preferably pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulfuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulfphonic, toluenesulphonic, benzenesulphonic, salicyclic, sulphanilic, aspartic, glutamic, edetic, stearic, palmitic, oleic, lauric, pantothenic, tannic, ascorbic and valeric acids.

The term “pro-drug” is used herein in its broadest sense to include those compounds which can be converted in vivo to the compound of interest (e.g. by enzymatic or hydrolytic cleavage). Examples thereof include esters, such as acetates of hydroxy or thio groups, as well as phosphates and sulphonates. Processes for acylating hydroxy or thio groups are known in the art, e.g. by reacting an alcohol (hydroxy group), or thio group, with a carboxylic acid. Other examples of suitable pro-drugs are described in Bundgaard, Design of Prodrugs, Elsevier 1985, the disclosure of which is included herein in its entirety by way of reference.

The term “metabolite” includes any compound into which the active agents can be converted in vivo once administered to the subject. Examples of such metabolites are glucuronides, sulphates and hydroxylates.

It will be understood that active agents as described herein may exist in tautomeric forms. The term “tautomer” is used herein in its broadest sense to include compounds capable of existing in a state of equilibrium between two isomeric forms. Such compounds may differ in the bond connecting two atoms or groups and the position of these atoms or groups in the compound. A specific example is keto-enol tautomerism.

The compounds of the present invention may be electrically neutral or may take the form of polycations, having associated anions for electrical neutrality. Suitable associated anions include sulfate, tartrate, citrate, chloride, nitrate, nitrite, phosphate, perchlorate, halosulfonate or trihalomethylsulfonate.

The active agents may be administered for therapy by any suitable route. It will be understood that the active agents are administered in one embodiment via a route that does not result in overt sedation of the subject, or result in dose-limiting side effects. Suitable routes of administration may include oral, rectal, nasal, inhalation of aerosols or particulates, topical (including buccal and sublingual), transdermal, vaginal, intravesical, parenteral (including subcutaneous, intramuscular, intravenous, intrasternal, intra-articular, injections into the joint, and intradermal) and intrathecal or epidural. In one embodiment, administration of the active agent is by a route resulting in first presentation of the compound to the stomach of the subject. In this embodiment, the active agents are generally administered via an oral route. In another embodiment the active agents are administered by the transdermal route. However, it will be appreciated that the route may vary with the condition and age of the subject, the nature of the pain being treated, its location within the subject and the judgement of the physician or veterinarian. It will also be understood that individual active agents may be administered by the same or different distinct routes. The individual active agents may be administered separately or together directly into a joint involved with an inflammatory painful process.

As used herein, an “effective amount” refers to an amount of active agent that provides the desired analgesic activity when administered according to a suitable dosing regime. The amount of active agent is generally an amount that provides the desired analgesic activity. In one aspect, this occurs, without causing overt sedation or dose limiting side-effects or drug tolerance. Dosing may occur at intervals of several minutes, hours, days, weeks or months. Suitable dosage amounts and regimes can be determined by the attending physician or veterinarian.

CNSB002 or pharmaceutically acceptable salts thereof, may be administered to a subject at a rate of between about 0.1 to about 50 mg/kg by body weight every from about 1 hour to up to about 50 hours, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 hours in amounts of 0.1, 0.2, 0.3, 0.4, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 mg/kg. Particularly useful times are from about 6 hours to about 24 hours, such as 6, 7, 8, 9, 10, 11, 12, 13, 14, 15, 16, 17, 18, 19, 20, 21, 22, 23, 24. Even more particular useful times are between from about 12 to about 24 hours. Such as 12, 13, 14, 15, 16, 17, 18, 19, 20, 21, 22, 23 or 24 hours. Dosing of the analgesic agent, such as an opioid, can be determined by the attending physician in accordance with dosing rates in practice. For example, fentanyl can be administered in an amount of about 100 μg whereas morphine may be administered in an amount of 10 mg, also on an hourly basis. The administration amounts may be varied if administration is conducted more or less frequently, such as by continuous infusion, by regular dose every few minutes (e.g. 1, 2, 3 or 4 minutes) or by administration every 5, 10, 20, 30 or 40 minutes (e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39 or 40 minutes) or every 1, 2, 3, 4, 5, 6, 7, 8, 9, 10, 11, 12, 13, 14, 15, 16, 17, 18, 19, 20, 21, 22, 23 or 24 hours or up to 50 hours such as, for example, 25, 26, 27, 28, 29, 30, 31, 32, 33, 34, 35, 36, 37, 38, 39, 40, 41, 42, 43, 44, 45, 46, 47, 48, 49 or 50 hours. In many instances, administration is conducted simply on the basis of when the patient requires pain relief.

Accordingly, a treatment protocol is contemplated for treating pain in a subject, the protocol comprising the steps of administration to the subject an effective amount of CNSB002 in conjunction with an opioid.

In another embodiment, a treatment protocol is provided for treating pain in a subject, the protocol comprising the steps of administration to the subject an effective amount of CNSB002 in conjunction with an opioid and/or an NK antagonist.

A further aspect also provides a composition comprising CNSB002 with an opioid and/or an NK antagonist together with one or more pharmaceutically acceptable additives and optionally other medicaments. The pharmaceutically acceptable additives may be in the form of carriers, diluents, adjuvants and/or excipients and they include all conventional solvents, dispersion agents, fillers, solid carriers, coating agents, antifungal or antibacterial agents, dermal penetration agents, surfactants, isotonic and absorption agents and slow or controlled release matrices. The active agents may be presented in the form of a kit of components adapted for allowing concurrent, separate or sequential administration of the active agents. Each carrier, diluent, adjuvant and/or excipient must be “pharmaceutically acceptable” in the sense of being compatible with the other ingredients of the composition and physiologically tolerated by the subject. The compositions may conveniently be presented in unit dosage form and may be prepared by methods well known in the art of pharmacy. Such methods include the step of bringing into association the active ingredient with the carrier, which constitutes one or more accessory ingredients. In general, the compositions are prepared by uniformly and intimately bringing into association the active ingredient with liquid carriers, diluents, adjuvants and/or excipients or finely divided solid carriers or both, and then if necessary shaping the product.

Compositions of the present invention suitable for oral administration may be presented as discrete units such as capsules, sachets or tablets each containing a predetermined amount of the active ingredient; as a powder or granules; as a solution or a suspension in an aqueous phase or non-aqueous liquid; or as an oil-in-water liquid emulsion or a water-in-oil emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. inert diluent, preservative disintegrant, sodium starch glycollate, cross-linked povidone, cross-linked sodium carboxymethyl cellulose) surface-active or dispersing agent. Moulded tablets may be made my moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for parenteral administration include aqueous and non-aqueous isotonic sterile injection solutions which may contain anti-oxidants, buffers, bacteriostats and solutes which render the composition isotonic with the blood of the intended subject; and aqueous and non-aqueous sterile suspensions which may include suspended agents and thickening agents. The compositions may be presented in a unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described. When reconstituted these can be in the form of aqueous solution, dissolved in water, isotonic saline or a balanced salt solution. Additionally, when reconstituted the product could be a suspension in which the compound(s) is/are dispersed in the liquid medium by combination with liposomes or a lipid emulsion such as soya bean.

Compositions suitable for topical administration to the skin, i.e. transdermal administration, may comprise the active agents dissolved or suspended in any suitable carrier or base and may be in the form of lotions, gels, creams, pastes, ointments and the like. Suitable carriers may include mineral oil, propylene glycol, waxes, polyoxyethylene and long chain alcohols. Transdermal devices, such as patches may also be used and may comprise a microporous membrane made from suitable material such as cellulose nitrate/acetate, propylene and polycarbonates. The patches may also contain suitable skin adhesive and backing materials.

The active compounds described herein may also be presented as implants, which may comprise a drug bearing polymeric device wherein the polymer is biocompatible and non-toxic. Suitable polymers may include hydrogels, silicones, polyethylenes and biodegradable polymers.

The compounds of the subject invention may be administered in a sustained (i.e. controlled) or slow release form. A sustained release preparation is one in which the active ingredient is slowly released within the body of the subject once administered and maintains the desired drug concentration over a minimum period of time. The preparation of sustained release formulations is well understood by persons skilled in the art. Dosage forms may include oral forms, implants and transdermal forms, joint injections, sustained or slow release injectables. For slow release administration, the active ingredients may be suspended as slow release particles or within liposomes, for example.

The compositions herein may be packaged for sale with other active agents or alternatively, other active agents may be formulated with CNSB002 or its pharmaceutical salts thereof and optionally an analgesic agent such as an opioid or NK antagonist. The composition may be sold or provided with a set of instructions in the form of a therapeutic protocol. This protocol may also include, in one embodiment, a selection process for type of patient or type of condition or a type of pain such as inflammatory or neuropathic pain.

Thus, a further aspect provides a system for the controlled release of active compounds selected from CNSB002 alone or in combination with an opioid and/or an NK antagonist or a pharmaceutically acceptable salt, derivative, homolog or analog thereof, alone or together with another analgesic or active agent, wherein the system comprises:

(a) a deposit-core comprising an effective amount of a first active compound and having defined geometric form, and

(b) a support-platform applied to the deposit-core, wherein the support-platform contains a second active compound, and at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

As used herein, the first active substance is one of (i) CNSB002, an opioid and/or an NK antagonist; or (ii) the other of CNSB002, an opioid and/or an NK antagonist. The second active substance may be (i) or (ii) above.

In another aspect, a system is provided for the controlled release for CNSB002 and an opioid, wherein the system comprises:

(a) a deposit-core comprising an effective amount of (1) omega conotoxin and (2) a neuronal excitation inhibitor, the deposit-core having a defined geometric form; and

(b) a support platform applied to the deposit-core, the support platform comprising at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

The support-platform may comprise polymers such as hydroxypropylmethylcellulose, plasticizers such as a glyceride, binders such as polyvinylpyrrolidone, hydrophilic agents such as lactose and silica, and/or hydrophobic agents such as magnesium stearate and glycerides. The polymer(s) typically make up 30 to 90% by weight of the support-platform, for example about 35 to 40%. Plasticizer may make up at least 2% by weight of the support platform, for example about 15 to 20%. Binder(s), hydrophilic agent(s) and hydrophobic agent(s) typically total up to about 50% by weight of the support platform, for example about 40 to 50%.

The tablet coating may contain one or more water insoluble or poorly soluble hydrophobic excipients. Such excipients may be selected from any of the known hydrophobic cellulosic derivatives and polymers including alkylcellulose, e.g. ethylcellulose, hydroxypropyl cellulose, hydroxypropylmethyl cellulose, carboxymethyl cellulose, and derivatives thereof; polymethacrylic polymers, polyvinyl acetate and cellulose acetate polymers; fatty acids or their esters or salts; long chain fatty alcohols; polyoxyethylene alkyl ethers; polyoxyethylene stearates; sugar esters; lauroyl macrogol-32 glyceryl, stearoyl macrogol-32 glyceryl, and the like. Hydroxypropylmethyl cellulose materials are preferably selected from those low Mw and low viscosity materials such as E-Type methocel, and 29-10 types as defined in the USP.

Other agents or excipients that provide hydrophobic quality to coatings may be selected from any waxy substance known for use as tablet excipients. Preferably they have a HLB value of less than 5, and more preferably about 2. Suitable hydrophobic agents include waxy substances such as carnauba wax, paraffin, microcrystalline wax, beeswax, cetyl ester wax and the like; or non-fatty hydrophobic substances such as calcium phosphate salts, e.g. dibasic calcium phosphate.

The coating may contain a calcium phosphate salt, glyceryl behenate, and polyvinyl pyrollidone, or mixtures thereof, and one or more adjuvants, diluents, lubricants or fillers.

Components in the coating may be as follows, with generally suitable percentage amounts expressed as percentage weight of the coating.

Polyvinyl pyrollidone (Povidone) is preferably present in amounts of about 1 to 25% by weight or the coating, more particularly 4 to 12%, e.g. 6 to 8%.

Glyceryl behenate is an ester of glycerol and behenic acid (a C22 fatty acid). Glyceryl behenate may be present as its mono-, di-, or tri-ester form, or a mixture thereof. Preferably it has an HLB value of less than 5, more preferably approximately 2. It may be present in amounts of about 5 to 85% by weight of the coating, more particularly from 10 to 70% by weight, and in certain preferred embodiments from 30 to 50%.

Calcium phosphate salt may be the dibasic calcium phosphate dihydrate and may be present in an amount of about 10 to 90% by weight of the coating, preferably 20 to 80%, e.g. 40 to 75%.

The coating may contain other common tablet excipients such as lubricants, colorants, binders, diluents, glidants and taste-masking agents or flavorants.

Examples of excipients include colorants such a ferric oxide, e.g. yellow ferric oxide; lubricants such as magnesium stearate; and glidants such as silicon dioxide, e.g. colloidal silicon dioxide. Yellow ferric oxide may be used in amounts of about 0.01 to 0.5% by weight based on the coating; magnesium stearate may be present in amounts of 1 to 20% by weight of the coating, more preferably 2 to 10%, e.g. 0.5 to 1.0%; and colloidal silica may be used in amounts of 0.1 to 20% by weight of the coating, preferably 1 to 10%, more preferably 0.25 to 1.0%.

The core comprises in addition to a drug substance, a disintegrating agent or mixtures of disintegrating agents used in immediate release formulations and well know to persons skilled in the art. The disintegrating agents useful in the exercise of the present invention may be materials that effervesce and or swell in the presence of aqueous media thereby to provide a force necessary to mechanically disrupt the coating material.

A core may contain, in addition to the drug substance, cross-linked polyvinyl pyrollidone and croscarmellose sodium.

The following is a list of contemplated core materials. The amounts are expressed in terms of percentage by weight based on the weight of the core.

Cross-linked polyvinyl pyrollidone is described above and is useful as a disintegrating agent, and may be employed in the core in the amounts disclosed in relation to the core.

Croscarmellose sodium is an internally cross-linked sodium carboxymethyl cellulose (also known as Ac-Di-Sol) useful as a disintegrating agent.

Disintegrating agents may be used in amounts of 5 to 30% by weight based on the core. However, higher amounts of certain disintegrants can swell to form matrices that may modulate the release of the drug substance. Accordingly, particularly when rapid release is required after the lag time it is preferred that the disintegrants is employed in amounts of up to 10% by weight, e.g. about 5 to 10% by weight.

The core may additionally comprise common tablet excipients such as those described above in relation to the coating material. Suitable excipients include lubricants, diluents and fillers, including but not limited to lactose (for example the mono-hydrate), ferric oxide, magnesium stearates and colloidal silica.

Lactose monohydrate is a disaccharide consisting of one glucose and one galactose moiety. It may act as a filler or diluent in the tablets of the present invention. It may be present in a range of about 10 to 90%, preferably from 20 to 80%, and in certain preferred embodiments from 65 to 70%.

The core should be correctly located within the coating to ensure that a tablet has the appropriate coating thickness.

In this way, lag times are reliable and reproducible, and intra-subject and inter-subject variance in bioavailability is avoided. It is advantageous to have a robust control mechanism to ensure that tablets in a batch contain cores having the appropriate geometry in relation to the coating. Controls can be laborious in that they require an operator to remove random samples from a batch and to cut them open to physically inspect the quality of the core (i.e. whether it is intact, and whether it is correctly located). Furthermore, if a significant number of tablets from the sample fail, a complete batch of tablets may be wasted. Applicant has found that if one adds to the core a strong colorant such as iron oxide, such that the core visibly contrasts with the coating when as strong light is shone on the tablet, it is possible for any faults in the position or integrity of the core to be picked up automatically by a camera appropriately located adjacent a tableting machine to inspect tablets as they are ejected therefrom.

Still another aspect provides a composition comprising: CNSB002; CNSB002 and an opioid; CNSB002 and an NK antagonist; or CNSB002, an opioid and an NK antagonist.

The composition also comprises one or more pharmaceutically acceptable carriers, excipients and/or diluents.

A method for the delivery of the composition to a subject is provided comprising the step of administering the composition to the subject orally, transdermally, or subdermally, wherein the composition comprises the components selected from above.

In one aspect, a tamper-proof narcotic delivery system is produced which provides for full delivery of narcotic medication and for analgesic action on legitimate patients while at the same time effectively eliminating the problem of tampering by diversion, adulteration, or pulverization of the medication for abuse by addicts. The compositions and methods herein are of value to those practiced in the medical arts and simultaneously possess no value or utility to individuals seeking to abuse or profit from the abuse of such analgesics.

It should be understood that in addition to the ingredients particularly mentioned above, the compositions herein may include other agents conventional in the art, having regard to the type of composition in question. For example, agents suitable for oral administration may include such further agents as binders, sweeteners, thickeners, flavoring agents, disintegrating agents, coating agents, preservatives, lubricants and/or time delay agents.

The formulation may also contain carriers, diluents and excipients. Details of pharmaceutically acceptable carriers, diluents and excipients and methods of preparing pharmaceutical compositions, and formulations are provided in Remmingtons Pharmaceutical Sciences 18^{th} Edition, 1990, Mack Publishing Co., Easton, Pa., USA.

In an embodiment, the active agents may also be presented for use in veterinary compositions. These may be prepared by any suitable means known in the art. Examples of such compositions include those adapted for:

(a) oral administration, e.g. drenches including aqueous and non-aqueous solutions or suspensions, tablets, boluses, powders, granules, pellets for admixture with feedstuffs, pastes for application to the tongue;

(b) parenteral administration, e.g. subcutaneous, intra-articular, intramuscular or intravenous injection as a sterile solution or suspension or through intra-nasal administration;

(c) topical application, e.g. creams, ointments, gels, lotions, etc.

In another embodiment, the active agents are administered orally, preferably in the form of a tablet, capsule, lozenge or liquid. The administered composition may include a surfactant and/or solubility improver. A suitable solubility improver is water-soluble polyethoxylated caster oil and an example of a suitable surfactant is Cremophor EL.

In one aspect, the opioid is morphine is administered at a rate and concentration of 100 micrograms/hour.

Mechanical devices are also provided for introduction to or in a body or body cavity coated with a sustained or slow release formulation of an omega conotoxin combined with the neuronal excitation inhibitor. Examples of mechanical devices include stents, catheters, artificial limbs, pins, needles, intrathecal implants and the like. Reference to an “intrathecal implant” includes reference to a cylindrical thread or device comprising a semipermeable membrane which permits passage or partial passage of small molecules (such as nutrients ad drugs in and cellular metabolic products out). The implant may also contain genetically modified or cultured cells (including stem cells) which secrete out useful cytokines and other metabolites. The implant may be designed to release molecules (or intake cellular by-products) for days, weeks, months or even years.

Stents, for example, typically have a lumen, inner and outer surfaces, and openings extending from the outer surface to the inner surface. The present invention extends to a method for coating a surface of a stent. At least a portion of the stent is placed in contact with a coating solution containing a coating material to be deposited on the surface of the stent. A thread is inserted through the lumen of the stent, and relative motion between the stent and the thread is produced to substantially remove coating material within the openings.

The thread can have a diameter substantially smaller than the diameter of the lumen. The thread can be inserted through the lumen either after or prior to contacting the stent with the coating solution. Relative motion between the stent and the thread can be produced prior to contacting the stent with the coating solution to clean the stent. The thread can be either a filament or a cable with a plurality of wires. The thread can be made of a metallic or polymeric material.

The stent can be dipped into the coating solution or spray coated with the coating solution. The coating material can include a biocompatible polymer, either with or without a pharmaceutically active compound.

In one embodiment, the relative motion is oscillatory motion produced by a vibrating device. The oscillations can be changed (magnitude and/or frequency) to vary thickness of the coating solution on the stent. In another embodiment, the relative motion is produced by a shaker table. Regardless of the type of motion, the relative motion can be produced either after or while the stent is in contact with the coating solution.

The relative motion between the stent and the thread can include initially moving the stent in a horizontal direction substantially parallel to the length of the thread and subsequently moving the stent in a vertical direction substantially perpendicular to the length of the thread. The movement in the horizontal direction can be repeated, with pauses between repetitions. The movement in the vertical direction can also be repeated, with the horizontal and vertical movements alternating.

In order to smooth the relative motion, the thread can be coupled to a damping compensator. The damping compensator connects the thread to a vibrating device. In one embodiment, the damping compensator comprises first and second filaments connected to the thread.

The relative motion can be motion of the stent along the thread. For example, a first end of the thread can be attached to a first stand at a first height and a second end of the thread is attached to a second stand at a second height. The relative motion is produced by a gravity gradient, with the first height differing from the second height. Furthermore, the stent can be moved back and forth between the first and second stands by sequentially increasing or decreasing at least one of the first and second heights. In this way, multiple coatings can be applied to the stent.

The relative motion can also be rotation of the stent relative to the thread. A stream of gas can be passed along at least a portion of the surface of the stent to rotate the stent relative to the thread. The rotation can also occur in conjunction with other relative motion between the stent and the thread.

An implantable medical device is also provided having an outer surface covered at least in part by CNSB002, CNSB002 and an opioid, CNSB002 and an NK antagonist or CNSB002, an opioid and an NK antagonist or pharmaceutically acceptable salts, derivative, homolog or analog thereof and optionally another active agent, a conformal coating of a hydrophobic elastomeric material incorporating an amount of active material therein for timed delivery therefrom and means associated with the conformal coating to provide a non-thrombogenic surface after the timed delivery of the active material.

In an embodiment, the conformal coating comprises an amount of finely divided biologically active material in the hydrophobic elastomeric material.

The present invention is now described with reference to the following Examples. Materials and Methods relevant to these Examples are described herein under.

All experiments were performed on male Wistar rats, weight 170-220 g for experiments investigating sedation and antinociception using the carrageenan inflammation paradigm and weight 120-200 g for experiments evaluating sedation and antinociception using the diabetic neuropathy model. The experiments were performed in an observer-blinded fashion with parallel positive placebo (gabapentin) and negative placebo (vehicle) treatment controls. All drug solutions and vehicle were given intraperitoneally (ip) in a volume of 1.0 ml.

Morphine sulfate (Mayne Pharma Pty Ltd), gabapentin (as 300 mg capsules, Arrow Pharmaceuticals Ltd), saline and sterile water (Pfizer) for injection were obtained from the pharmacy at Monash Medical Centre, Monash University, Clayton, Victoria, Australia. Streptozotocin was obtained from Sapphire Bioscience Pty ltd, (Suite 1, 134 Redfern Street, Redfern, New South Wales 2016, Australia). Carrageenan was Type I (Sigma-Aldrich Chemical Company, PO Box 970, Castle Hill, New South Wales 1765, Australia). CNSB002 [formerly AM36] was obtained from AMRAD, later known as Zenyth Therapeutics Ltd (576 Swan Street, Richmond, Victoria 3121, Australia) as a di-L-tartrate salt. Its chemical name is 1-(2-(4-chlorophenyl)-2-hydroxy)ethyl-4-(3,5-bis(1,1-dimethyl)-4-hydroxyphenyl)methyl-piperazine. Male Wistar rats were supplied as specified pathogen free HsdBrlHan:WIST strain by Monash University Animal Services, Clayton, Victoria, Australia. All rats were housed in high topped cages in groups of four per cage in a temperature controlled room with a 12 hour light/dark cycle. The rats were allowed food and water ad-libitum.

Prior to investigation of the antinociceptive effects of morphine and CNSB002, experiments were performed to define the doses of each drug given alone and in combination that did not cause any signs of sedation or CNS depression in any rats. Measurement of the movements of a freely moving rat in an open field activity monitor was used. Each rat was subjected to this test on only two occasions on successive days to avoid habituation to the test; only one of those days involved active drug or placebo, the other being drug vehicle control. The maximum non-sedating doses of each compound alone and in combination were first determined in normal rats and then those doses were tested for sedation in diabetic rats. This was done to be sure that there was no sedation caused by the drugs, particularly in diabetics that are known to be more susceptible to CNS depressants. Only doses of compounds found not to be sedating alone or in combinations were tested for antinociceptive effects in either model.

The open field activity monitor (MedAssociates Inc. St. Albans, Vt. 05478, USA) is an enclosure with a quiet and darkened environment. It contains 16 intersecting beams of infrared light. Interruptions of beams by individual rats moving around the enclosure were recorded by computer. These measurements were converted to the total rest time in seconds during a 20 min period in the apparatus. Rest times of drug-treated rats were compared with vehicle-treated controls using ANOVA with Dunnet post hoc test.

Animals naïve to drug treatment were acclimatized to the lab environment and individual rats were selected randomly from a group one at a time. They were injected (ip) with vehicle, a test drug dose or a combination of drugs in an operator-blinded fashion. Five minutes after the ip injection each rat was placed in the open field activity monitor for a 20-min period.

Groups of rats were tested with the open field activity monitor as above with the following ip treatments:
vehicle controls, n=121 experiments
CNSB002 at doses 10 and 20 mg/kg, n=20 and 10 experiments
morphine at doses 6.4 and 3.2 mg/kg, n=21 and 20 experiments
a combination of CNSB002 at 10 mg/kg with morphine at 3.2 mg/kg, n=21 experiments
a combination of CNSB002 at 5.0 mg/kg with morphine at 3.2 mg/kg, n=19 experiments
gabapentin at doses of 100 and 50 mg/kg, n=26 each experiments
a combination of GABApentin at 50 mg/kg with morphine at 3.2 mg/kg, n=19 experiments

Two tests for antinociception were used: the carrageenan paw inflammation test; and the streptozotocin-induced diabetic neuropathy model. Inflammation of the right hind paw was induced by an intraplantar injection of carrageenan (100 μl of a 2% w/v carrageenan solution in saline). Time was allowed for paw inflammation to develop (2 hours). Paw withdrawal latencies were measured using noxious heat from an infrared beam focused onto the plantar surface of the right hind paw in unrestrained animals using apparatus from Ugo Basile (via G. Borghi 43, 21025 Comerio VA—Italy). The method used was originally described by Hargreaves et al, Pain 32:77-88, 1988.

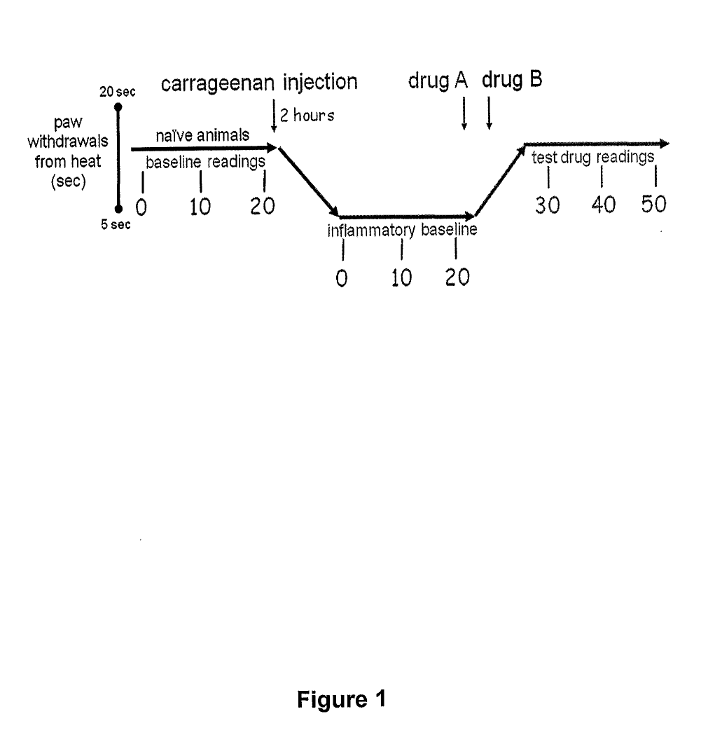

Paw withdrawal latencies were measured at 10 min intervals before the induction of inflammation with carrageenan injection until 3 stable baseline readings were obtained (baseline readings—see FIG. 1). Once an inflammatory reaction was initiated 2 hours after the carrageenan injection, paw withdrawal thresholds were measured again three times at 10 min intervals (inflammatory baseline—see FIG. 1) to confirm the development of hyperalgesia; a decrease in paw withdrawal latency typically from control pre-carrageenan level of 12 seconds to 6 seconds. A test drug or drug combination was injected intraperitoneally and paw withdrawal values were measured at 10-minute intervals for the following 30 minutes (test drug readings—see FIG. 1).

The following drug treatments were given ip to separate groups of rats:
vehicle controls, n=63
CNSB002 at dose range 0.5 mg/kg to 10 mg/kg alone, n=14 each
morphine at dose range 0.2 mg/kg to 3.2 mg/kg alone, n=10 each
combinations of CNSB002 at 5 mg/kg with morphine at 0.2, 0.4, 0.8, 1.6 and 3.2 mg/kg, n=10 each
gabapentin alone at 50 mg/kg, n=20
combination of gabapentin at 50 mg/kg with morphine 3.2 mg/kg, n=20

$$\% Reversal Hyperalgesia = \frac{\{ \begin{matrix}mean of test drug readings - \\ mean of inflammatory baseline\end{matrix} \}}{\{ \begin{matrix}mean of baseline readings - \\ mean of inflammatory baseline\end{matrix} \}} \times 100 \% \qquad Equation 1$$

The values for percent reversal of hyperalgesia were calculated as shown in Equation 1 for each rat and then results from animals treated similarly (e.g. vehicle and gabapentin controls, CNSB002 alone, morphine alone, and drug combinations) were combined to calculate means±SEM that were plotted as dose response curves. Drug and placebo/vehicle treatments were compared statistically using ANOVA with Tukey-Kramer post hoc test.

The methods used in this model were described previously by Courteix et al, Pain 53:81-88, 1993; Courteix et al, Pain 57(2):153-160, 1994, but were modified for use with a heat nociception test. Male Wistar rats weight 65-85 g were used.

Rats were injected ip with streptozotocin (STZ; 160 mg/kg total dose) dissolved in 0.9% w/v sodium chloride solution. The 150 mg dose was given in two 75 mg/kg injections on consecutive days. Diabetes was confirmed one week after injection of STZ by measurement of tail vein blood glucose levels with AccuCheck Active test strips and a reflectance colorimeter (AccuCheck Glucometer, Roche). Only animals with final blood glucose levels ≧15 mM were deemed to be diabetic. The rats were retested for hyperglycaemia once per week to confirm continued high blood glucose readings. Hyperalgesia was assessed by measurement of paw flick latency using the radiant heat plantar test described previously by Hargreaves et al, 1988 supra.

Tests of antinociception took place 5 weeks after the first injection of STZ. Animals that had a paw flick latency equal to or below 7.0 seconds (at least 40% of the value in normal weight-matched rats) were deemed to have developed hyperalgesia/neuropathic pain and thus used in further experiments to assess drug effects (50% of STZ-treated rats). Up to five experiments involving drug or placebo injection were performed on each diabetic hyperalgesic rat, one per day on successive days.

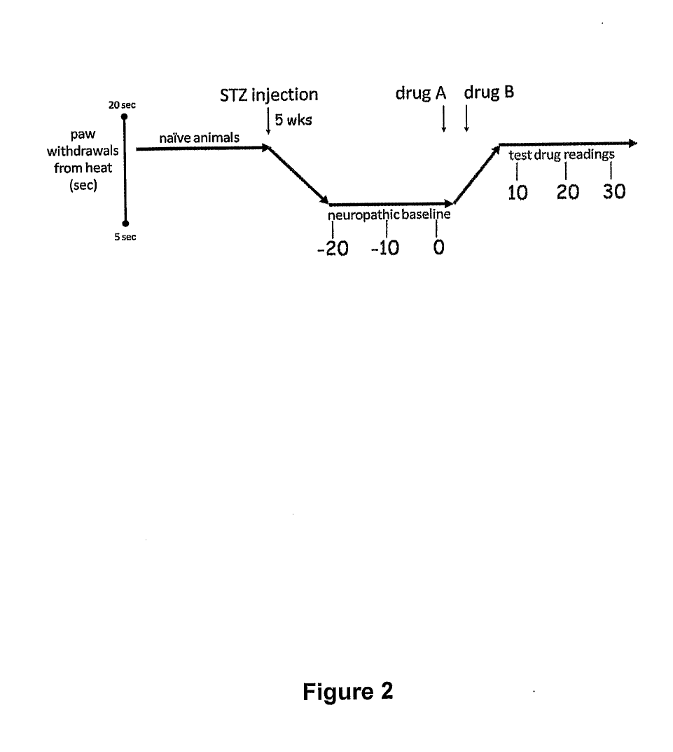

After the development of hyperalgesia in diabetic animals was confirmed by the radiant heat plantar test, more detailed nociceptive testing paradigms were carried out in diabetic neuropathic animals and weight-matched controls (wt 120-200 g); the control rats were 1-2 weeks younger. Historical controls (a threshold measured in a young (wt 80 g) before STZ treatment) were not used for comparison with thresholds following diabetes induction and drug tests performed 5 weeks later. The weight-matched control animals were chosen rather than similar aged rats to reflect the change in paw plantar fat that can alter the tissue heat conduction and, therefore, the observed paw withdrawal latency. Paw flick latency (PFL) was measured by the method described by Hargreaves et al, 1988 supra using the Ugo-Basile Plantar Test Apparatus (IR 50); an intense thermal stimulus was applied to the right hind paw until paw withdrawal was elicited or a maximum cut-off time of 23 s was reached. Paw withdrawal thresholds were measured as shown in FIG. 2 in groups of rats 20 minutes and 10 minutes before, immediately before (time 0) and also at 10, 20 and 30 minutes after ip injections of:
vehicle controls n=39 experiments
weight matched non diabetic controls (no treatment), n=41 experiments
CNSB002 at dose range 0.5 mg/kg to 10 mg/kg alone, n=15 each experiments
morphine at dose range 0.2 mg/kg to 3.2 mg/kg alone, n=16 each experiments
gabapentin 50 mg/kg alone, n=17 experiments
combinations of CNSB002 at 5 mg/kg with morphine at 0.2, 0.4, 0.8, 1.6 and 3.2 mg/kg, n=14 each experiments
gabapentin 50 mg/kg plus morphine 3.2 mg/kg together, n=26 experiments

The percentage reversal of hyperalgesia (calculated as shown in Equation 2) in each experiment was combined with other replicate values in rats treated similarly (vehicle and gabapentin controls, morphine alone, CNSB002 alone and drug combinations) to calculate means±SEM. These were plotted as dose response curves. Drug and placebo/vehicle treatments were compared statistically using ANOVA with Tukey-Kramer post hoc test.

$$\% Reversal Hyperalgesia = \frac{\{ \begin{matrix}mean of test drug effect - \\ mean of neuropathic baseline\end{matrix} \}}{\{ \begin{matrix}mean of weight \text{-} matched rats baselines - \\ mean of neuropathic baseline\end{matrix} \}} \times 100 \% \qquad Equation 2$$

### EXAMPLE 1

### Carrageenan-Induced Inflammation and Sedation

A total of 344 rats were used for studies of carrageenan-induced inflammation and sedation (170-220 g weight range). A total of 164 diabetic animals were used (120-200 g weight range) to investigate antinociceptive properties of drugs alone and in combination as well as sedation.

### EXAMPLE 2

### Open Field Activity Monitor

Tables 1A,B and C show the results from the open field activity monitor test for normal animals. The values marked with an asterisk and in bold are statistically significantly different when compared with vehicle treated animals (one way ANOVA with Dunnett post hoc correction). It can be concluded from this set of experiments that sedation was not caused in these rats by doses of morphine up to 6.4 mg/kg ip. In addition doses of CNSB002 greater than 10 mg/kg ip when administered alone as well as combinations of CNSB002 greater than 5 mg/kg with morphine at 3.2 mg/kg (*; p<0.001) caused sedation. Gabapentin doses greater than 50 mg/kg were found to cause sedation in normal rats when administered alone (*; p<0.01). However, when combined with morphine at 3.2 mg/kg gabapentin 50 mg/kg did not cause sedation.

Results of sedation testing in diabetic animals are shown in tables 2A and B. With the exception of morphine 6.4 mg/kg the doses of the drugs that did not cause sedation in normal rats also did not cause any sedation in diabetics. Therefore, the highest doses of CNSB002 and morphine investigated for antinociceptive effects were:
morphine 3.2 mg/kg; ip alone
CNSB002 10 mg/kg; ip alone
Morphine 3.2 mg/kg; ip in combination with CNSB002 5 mg/kg ip
Gabapentin 50 mg/kg; ip alone
Gabapentin 50 mg/kg; ip in combination with morphine 3.2 mg/kg ip

### EXAMPLE 3

### Carrageenan Paw Inflammation

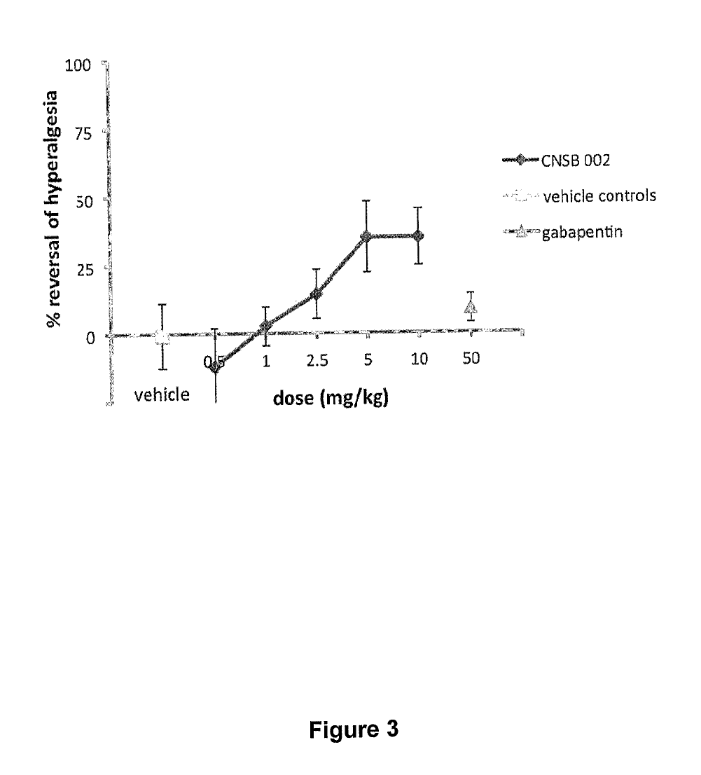

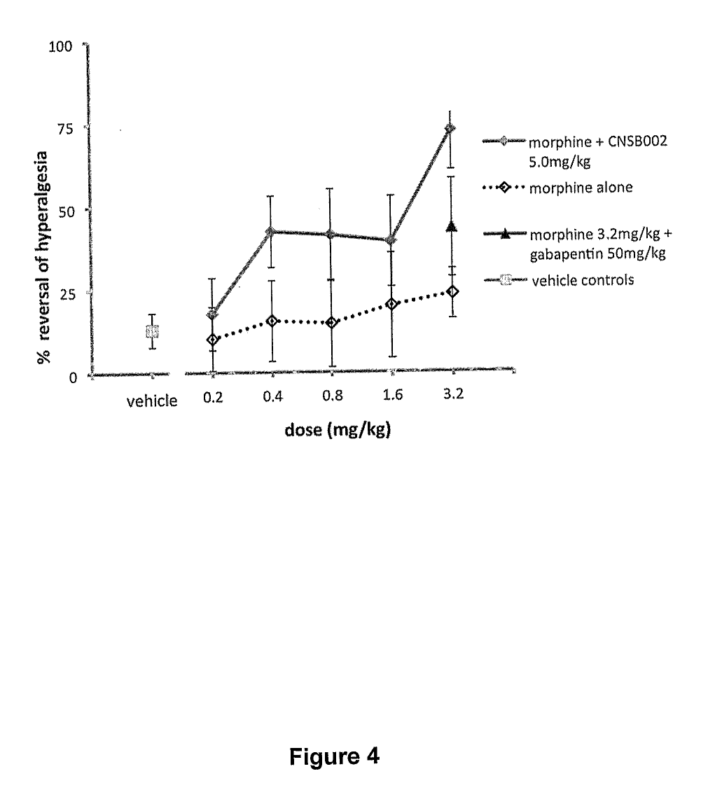

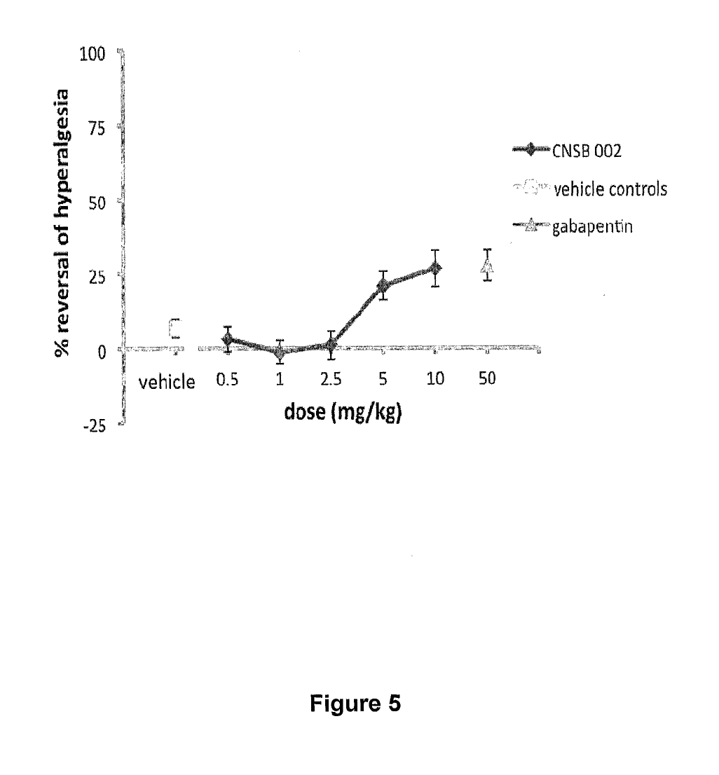

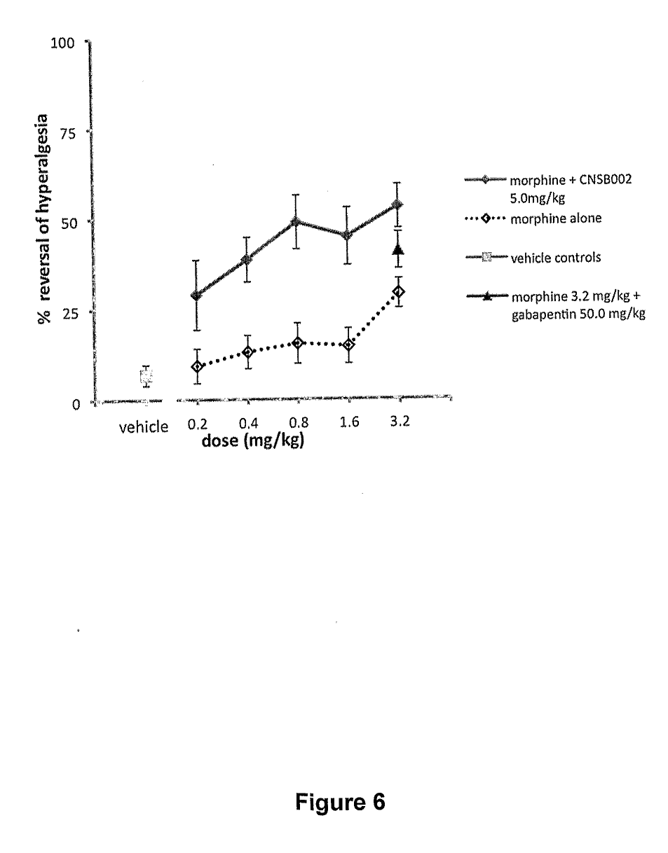

Dose response curves for reversal of carrageenan-induced hyperalgesia by CNSB002 given alone and in combination with morphine are shown in FIGS. 3 and 4. Points shown are means and bars SEM. CNSB002 administered alone at its highest non-sedating doses of 5 and 10 mg/kg caused a moderate effect on carrageenan-induced nociceptive behaviour, both reversing the hyperalgesia by 35%. No dose of gabapentin or CNSB002 administered alone caused statistically-significant antinociception in carrageenan paw inflammation (p>0.05; one way ANOVA with Tukey-Kramer post hoc test; comparison with vehicle controls). Morphine administered alone also caused no significant antinociception in, the carrageenan paradigm (p>0.05; one way ANOVA with Tukey-Kramer post hoc test; comparison with vehicle controls). Morphine at 3.2 mg/kg administered alone caused only 24% reversal of carrageenan-induced hyperalgesia, but when administered together with CNSB002 at 5 mg/kg it caused a significant antinociceptive effect, reversing inflammatory hyperalgesia in the right paw by 73%; a threefold increase above the antinociceptive effect of morphine given alone in this model (p<0.001; one way ANOVA with Tukey-Kramer post hoc correction). Morphine 3.2 mg/kg administered together with gabapentin 50 mg/kg also resulted in increased antinociceptive effect in the carrageenan paradigm, being 43% reversal of carrageenan-induced hyperalgesia.

### EXAMPLE 4

### Streptozotocin (STZ)-Induced Diabetic Neuropathy

Diabetic neuropathy caused an average 48% reduction in baseline nociceptive thresholds in diabetic rats compared with normal weight matched controls (FIG. 5). The dose response curves for each mode of treatment in rats with hyperalgesia caused by diabetic neuropathy are shown in FIGS. 5 and 6. Points shown are means and bars SEM. CNSB002 when administered alone at the dose of 10 mg/kg caused significant antinociceptive effect in diabetic neuropathy and was similar to the effect of gabapentin at 50 mg/kg alone (FIG. 5). Both treatments caused 26% reversal of hyperalgesia (p<0.01; one way ANOVA with Tukey-Kramer post hoc test; comparison with vehicle controls). No other doses of CNSB002 administered alone caused significant antinociception.

One dose of morphine administered alone caused significant antinociceptive effect in streptozotocin (STZ)-induced diabetic neuropathy (3.2 mg/kg; 30% reversal of hyperalgesia; p<0.05—one way ANOVA with Tukey-Kramer post hoc test; comparison with vehicle controls). This effect of morphine was increased significantly (53% reversal of diabetes-induced hyperalgesia) by co-administration with 5 mg/kg CNSB002 (FIG. 6; comparison with vehicle controls—p<0.001; one way ANOVA with Tukey-Kramer post hoc test). Similarly, gabapentin at 50 mg/kg administered together with morphine at 3.2 mg/kg caused 41% reversal of hyperalgesia; this was statistically significant (p<0.001; one way ANOVA with, Tukey-Kramer post hoc test). However, statistical analysis revealed that the effect of the combination of gabapentin at 50 mg/kg with 3.2 mg/kg morphine was not significantly different from any doses of morphine administered alone. By contrast, the antinociception following co-administration of 5 mg/kg CNSB002 with morphine at two doses, 3.2 and 0.8 mg/kg was significantly greater then morphine administered at those doses alone (p<0.05; one way ANOVA with Tukey-Kramer post hoc test).

TABLE 1A
normal rats open field activity monitor

| Treatment | mean | SEM | N |
|---|---|---|---|
| vehicle controls | 804.1 | 20.6 | 26 |
| CNSB002 20 mg/kg alone | 1001.2* | 37.7 | 10 |
| CNSB002 10 mg/kg alone | 851.2 | 30.9 | 10 |
| CNSB002 10 mg/kg plus morphine 3.2 mg/kg in | 998.1* | 31.6 | 10 |
| combination | | | |
| CNSB002 5 kg plus morphine 3.2 mg/kg in | 826.0 | 44.0 | 10 |
| combination | | | |

One way ANOVA with Dunnett post hoc test: CNSB002 20 mg/kg, CNSB002 10 mg/kg plus morphine 3.2 mg/kg were sedating in normal rats (*; p<0.001). CNSB002 at dose 10 mg/kg did not cause sedation when administered alone (p>0.05). The combinations of morphine 3.2 mg/k g with CNSB002 5 mg/k g was also not sedating (p>0.05).

TABLE 1B
normal rats open field activity monitor

| Treatment | mean | SEM | N |
|---|---|---|---|
| vehicle controls | 960.0 | 45.0 | 10 |
| morphine 3.2 mg/kg alone | 961.0 | 37.0 | 10 |

Morphine 3.2 mg/kg administered alone did not cause sedation in normal rats p>0.05; (unpaired t test).

TABLE 1C
normal rats open field activity monitor

| Treatment | mean | SEM | N |
|---|---|---|---|
| vehicle controls | 720.9 | 21.5 | 10 |
| gabapentin 100 mg/kg alone | 881.6* | 47.0 | 13 |
| gabapentin 50 mg/kg alone | 753.0 | 33.8 | 13 |
| gabapentin 50 mg/kg plus morphine 3.2 | 864.0 | 33.1 | 8 |
| mg/kg in combination | | | |

Gabapentin at dose 50 mg/kg administered alone or in combination with morphine 3.2 mg/kg did not cause sedation in normal animals (p>0.05; one way ANOVA with Dunnett post hoc test). In contrast, gabapentin 100 mg/kg alone caused significant sedation (*; p<0.01).

TABLE 2A
diabetic rats open field activity monitor

| Treatment | mean | SEM | N |
|---|---|---|---|
| vehicle controls | 929.8 | 10.3 | 57 |
| gabapentin 100 mg/kg alone | 941.2 | 22.6 | 13 |
| gabapentin 50 mg/kg alone | 985.8 | 26.4 | 13 |
| CNSB002 10 mg/kg alone | 1005.6 | 31.3 | 10 |
| morphine 3.2 mg/kg alone | 943.6 | 31.9 | 10 |

Gabapentin (100 and 50 mg/kg), CNSB002 (10 mg/kg) and morphine (3.2 mg/kg) were not sedating in diabetic rats when given alone (p>0.05; one way ANOVA with Dunnett post hoc test)

TABLE 2B
diabetic rats open field activity monitor

| Treatment | mean | SEM | N |
|---|---|---|---|
| vehicle controls | 888.6 | 19.2 | 18 |
| gabapentin 50 mg/kg plus morphine 3.2 | 921.5 | 35.8 | 11 |
| mg/kg in combination | | | |
| CNSB002 10 mg/kg plus morphine 3.2 mg/kg | 1114.1* | 27.8 | 11 |
| in combination | | | |
| CNSB002 5 mg/kg plus morphine 3.2 mg/kg | 976.1 | 24.6 | 9 |
| in combination | | | |
| morphine 6.4 mg/kg alone | 972.6* | 14.8 | 21 |

One way ANOVA with Dunnett post hoc test: Morphine 6.4 mg/kg alone and CNSB002 10 mg/kg plus morphine 3.2 mg/kg caused sedation in diabetic rats (*; p<0.01). The combinations of morphine 3.2 mg/kg with CNSB002 5 mg/kg or gabapentin 50 mg/kg were not sedating (p>0.05).

Those skilled in the art will appreciate that the invention described herein is susceptible to variations and modifications other than those specifically described. It is also to be understood that the invention includes all such variations and modifications. The present invention also includes all steps, features, compositions and compounds referred to, or indicated in this specification, individually or collectively, and any and all combinations of any two or more of said steps or features.

### BIBLIOGRAPHY

Backonja, Anesth Analg 97:785-790, 2003
Bundgaard, Design of Prodrugs, Elsevier 1985
Catterall, Trends Pharmacol Sci 8:57-65, 1987
Chabal et al, Anesthesiology 76(4):513-517, 1992
Chapman et al, Br J Pharmacol 116:1628-1634, 1995
Chonjnowska, Anesth Analg 90:1007-1008, 2000
Chou-Tan et al, Am J Phys Med Rehabil 75:84-87, 1996
Courteix et al, Pain 53:81-88, 1993
Courteix et al, Pain 57(2):153-160, 1994
Deffois et al, Neurosci Lett 220(2):117-120, 1996
Dejgard et al, Lancet 1 (8575-6):9-11, 1988
Eisenberg et al, Neurology 57:505-509, 2001
Hargreaves et al, Pain 32:77-88, 1988
Jarrott et al, Drug Development Research 46:261-267, 1999
Kajander and Bennett, J. Neurophysiol 68(3):734-744, 1992
Kajander et al, Neurosci Lett 138(2):225-228, 1992
Kalso et al, Eur J Pain 2(1):3-14, 1998
Kastrup et al, Pain 28(1):69-75, 1987
MacDonald, Curr Opin Neurol 10:121-128, 1997
Max et al, Neurology 37(4):589-596, 1987
Suzuki, Neuroreport 11 (12):R17-R21, 2000
Tremont-Lukats et al, Anesth Analg 101:1738-1749, 2005
Woolf, Nature 306:686-688, 1983

1. A method for inducing an analgesic response to inflammatory or neuropathic pain in a subject, comprising the administration to a subject of an amount of a compound, CNSB002, comprising the structure 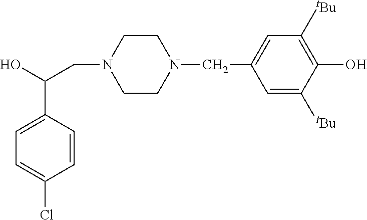
either alone or in combination with an opioid and/or an NK antagonist.

2. The method of claim 1 wherein the NK antagonist is selected from achiral pyridine class of neurokinin-1 receptor antagonists; netupitant 21; betctupitant 29; elzlopitant; lanepitant; osanetant; talnetant; GR205171; MK 0517; MK517; MEN 11467; nepadutant; MEN 11420; M274773; [Sar (9), Met (02) (11)]-Substance P; Tyr (6), D-Phe (7), D-His (9)-Substance-P (6-11) (sendide); (beta;-Ala(8))-Neurokinin A (4-10); (Tyr(5), D-Trp (6,8,9), Lys-NH(2) (10))-Neurokinin A; [D-Proz, D-Trip 7,9]-SP DPDT-SP; [D-Proz, D-Phe7, D-Trp9]-SP; SR48968 and 4-Alkylpiperidine derivative; telnetant; SB223412; SB223412A; telnetant hydrochloride; MDL103392; phosphorylated morpholine acetal human neurokinin-1 receptor agonists; SDZ NKT 343; LY 303 870; Ym-35375 and spiro-substituted piperidines; YM-44778; YM-38336; Septide; L732,13; Dactinomyan analogues; MEN 10207; L 659874; L 668,169; FR113680 and derivative; GR 83074; tripeptides possersi, the glutaminyl-D-trypto phy phenyl alonite sequence; L 659,877; R396; Imidazo[4,5-b]quinoxaline cyonines as neurokinin antagonists; MEN 10208; DPDTP-octa; GR73632; GR64349; senktide; GR71251; [D-Arg1, D-Pro2, D-Trp 7,9, Leu11]-SP (1-11); Ac heu-Asp-Gln-Trp-Phe-Gly NH2; Thr-Asp-Tyr-D-Tvp-Val-D-Trp-D-Trp-Arg NH2; Cyclo [Eln-Trp-Phe-Gly-Leu-Met]; D-Pro2D-Trp 7,9; D-Arg1D-Trp 7,9 leu11; [Gly6]-NKB [3-10]; [Arg3, D-Ala6]-NKB [3-10]; CP-9634; 3 aminoquinudidine; CP-99994; S18525; S19752; 4-quinoline carboxinide fremincik class; CP-122721; MK-869; GR205171; Spantide II; CP-96,345; L703,606; SR140, DNK333; 2-phenyl-4-quinolinecarboximides class; FK224; FR 115224; FK888; ZM253270-pyrrolopyrimidine class of nonpeptide neurokinin antagonists; GR71251; GR82334; RP67580; diacylpiperazine antagonists of human neurokinin eg L-161664; RP67580; MEN10376; GR98400; N2-[N2-(1H-indol-3-ylcarbonyl)-L-lysyl]-N-methyl-N-(phenyl-methyl)-L-phenylalaninamibe (2b); SP-(1-11); SP-(6-11); SP-(4-11) WIN51703; Spantide II; Spantide III; Spantide I; aprepitant; L754030; MK0869; ONO-7436; ONO 7436; MEN13510; 1-[2-(R)-{1-1R)-[3,5-bis(trifluoromethyl)phenyl]ethoxy}-3-(R)-(3,4-difluorophenyl)-4-(R)-tetrahydro-2H-pyran-4-ylmethyl]-3-(r)-methylpiperdine-3-carboxylic acid (1); LY 306,740; SLV-323; 2-substituted-4-aryl-6,7,8,9-tetrahydro-5H-pyrimido[4,5-b][1,5]oxazocin-5-one; 9-substituted-7-aryl-3,4,5,6-tetrahydro-2H-pyrido[4,3-b]- and [2,3-b]-1,5-oxazocin-6-one; SR142801; SB222200; CP96345; SR48968; ezlopitant; CJ 11974; MEN11558; [18F] SPA-RQ; neuropitant 21; betupitant 29; SR 144190; SR48692; SR141716; L733060; vofopitant; R-673; nepadutant; saredutant; UK 290795; 2-(4-biphenylyl)quinoline-4-carboxylate and carboxamide analogs (neurokinin-3 receptor antagonist); 4-Amino-2-(aryl)-butylbenzamides and analogues; MK-869; L742694; CP 122721; 1-alkyl-5-(3,4-dichlorophenyl)-5-[2-[(3-substituted)-1-azetidinyl]ethyl]-2-piperidines; L760735; L758,298, Cbz-Gly-Leu-Trp-0Bzl(CF(3))(2); L733,061; SR144190; SB235375; N—[(R,R)-(E)-1-arylmethyl-3-(2-oxo-azepan-3-yl) carbamoyl]allyl-N-methyl-3,5-bis(trifluoromethyl)benzamides; 3-[N¹-3,5-bis (trifluoromethyl)benzoyl-N-arylmethyl-N¹-methylhydrazino]-N—[(R)-2-oxo-azepan-3-yl]propionanides; SR142806; SR48,968; CP141,938; LY306740; SB40023; SB414240; Nolpitantium; SR140333; perhydroisoindole RP 67580, Depitant; RPR 100893; Lanepitant; LY-303870; sanoti synthelabo; nolpitanium; SR 140333; SR 48968; Savedutant; AV 608; AV-608, AV608; CGP 60829; NK-608; NKP-608C; NKP608; CS003; R113281; Vestipitant; 597599; GW 597599; GW 597599B; SSR 240600; casopitant; 679769; GW 679769; TA 5538; SSR 146977; SLV317; SLV-317; 823296; GW 823296; AVE 5883; AVE-5883; AZ 311; SB 235375; SB 733210; AZ 685; SAR 102279; SAR 10279; SSR 241586; SLV 332; Neurokinin 2 antagonist-Solvay; SLV-332; SLV332, NIK 616; MPV4505; NIK616; MPC 4505; Z501; Z-501; 1 TAK 637; CP 96345; L 659877; CGP 49823; GR 203040; L 732138; S 16474; WIN 51708; ZD 7944; S 18523; CI 1021; PD 154075; 758298; ZD 4974; S 18920; HMR 2091; FK 355; SCH 205528; NK 5807; NIP 531; SCH 62373; UK 224671; MEN 10627; WIN 64821; MDL 105212A; MEN 10573; TAC 363; 1 MEN 11149; HSP 117; NIP 530; and AZD 5106.

3. The method of claim 2, wherein the NK antagonist is aprepitant.

4. The method of claim 1 wherein the opioid is selected from morphine, fentanyl, oxycodone, codeine, dihydrocodeine, dihydrocodeinone enol acetate, desomorphine, apomorphine, diamorphine, pethidine, methadone, dextropropoxyphene, pentazocine, dextromoramide, oxymorphone, hydromorphone, dihydromorphine, papverine, papveretum, alfentanil, buprenorphine, tramadol and an opioid agonist and pharmaceutically acceptable derivates, homologs or analogs thereof.

5. The method of claim 1 wherein CNSB002 is administered in combination with an opioid.

6. The method of claim 5 wherein the opioid is morphine.

7. The method of claim 1 wherein CNSB002 is administered in combination which an NK antagonist.

8. The method of claim 1 wherein the subject is human.

9. A delivery system for inducing an analgesic response in a subject having inflammatory or neuropathic pain said delivery system comprising combined or separate formulations of (1) CNSB002; (2) two or more compounds selected from CNSB002, an opioid and/or an NK antagonist; and optionally (3) one or more further active agents or pharmaceutically acceptable carriers, excipients or diluents.

10. A method of treating inflammatory or neuropathic pain associated with a disease or physiological condition in a subject, said method comprising:
(a) selecting the subject on the basis of the subject having inflammatory or neuropathic pain;
(b) administering to said subject identified in step (a) an effective amount of CNSB002 either alone or in combination with an opioid and/or an NK antagonist.

11. The method of claim 10 wherein the NK antagonist is selected from achiral pyridine class of neurokinin-1 receptor antagonists; netupitant 21; betctupitant 29; elzlopitant; lanepitant; osanetant; talnetant; GR205171; MK 0517; MK517; MEN 11467; nepadutant; MEN 11420; M274773; [Sar (9), Met (02) (11)]-Substance P; Tyr (6), D-Phe (7), D-His (9)-Substance-P (6-11) (sendide); (beta;-Ala(8))-Neurokinin A (4-10); (Tyr(5), D-Trp (6,8,9), Lys-NH(2) (10))-Neurokinin A; [D-Proz, D-Trip 7,9]-SP DPDT-SP; [D-Proz, D-Phe7, D-Trp9]-SP; SR48968 and 4-Alkylpiperidine derivative; telnetant; SB223412; SB223412A; telnetant hydrochloride; MDL103392; phosphorylated morpholine acetal human neurokinin-1 receptor agonists; SDZ NKT 343; LY 303 870; Ym-35375 and spiro-substituted piperidines; YM-44778; YM-38336; Septide; L732,13; Dactinomyan analogues; MEN 10207; L 659874; L 668,169; FR113680 and derivative; GR 83074; tripeptides possersi, the glutaminyl-D-trypto phy phenyl alonite sequence; L 659,877; R396; Imidazo[4,5-b]quinoxaline cyonines as neurokinin antagonists; MEN 10208; DPDTP-octa; GR73632; GR64349; senktide; GR71251; [D-Arg1, D-Pro2, D-Trp 7,9, Leu11]-SP (1-11); Ac heu-Asp-Gln-Trp-Phe-Gly NH2; Thr-Asp-Tyr-D-Tvp-Val-D-Trp-D-Trp-Arg NH2; Cyclo [Eln-Trp-Phe-Gly-Leu-Met]; D-Pro2D-Trp 7,9; D-Arg1D-Tip 7,9 leu11; [Gly6]-NKB [3-10]; [Arg3, D-Ala6]-NKB [3-10]; CP-9634; 3 aminoquinudidine; CP-99994; S18525; S19752; 4-quinoline carboxinide fremincik class; CP-122721; MK-869; GR205171; Spantide II; CP-96,345; L703,606; SR140, DNK333; 2-phenyl-4-quinolinecarboximides class; FK224; FR 115224; FK888; ZM253270-pyrrolopyrimidine class of nonpeptide neurokinin antagonists; GR71251; GR82334; RP67580; diacylpiperazine antagonists of human neurokinin eg L-161664; RP67580; MEN10376; GR98400; N2-[N2-(1H-indol-3-ylcarbonyl)-L-lysyl]-N-methyl-N-(phenyl-methyl)-L-phenylalaninamibe (2b); SP-(1-11); SP-(6-11); SP-(4-11) WIN51703; Spantide II; Spantide III; Spantide I; aprepitant; L754030; MK0869; ONO-7436; ONO 7436; MEN13510; 1-[2-(R)-{1-1R)-[3,5-bis(trifluoromethyl)phenyl]ethoxy}-3-(R)-(3,4-difluorophenyl)-4-(R)-tetrahydro-2H-pyran-4-ylmethyl]-3-(r)-methylpiperidine-3-carboxylic acid (1); LY 306,740; SLV-323; 2-substituted-4-aryl-6,7,8,9-tetrahydro-5H-pyrimido[4,5-b][1,5]oxazocin-5-one; 9-substituted-7-aryl-3,4,5,6-tetrahydro-2H-pyrido[4,3-b]- and [2,3-b]-1,5-oxazocin-6-one; SR142801; SB222200; CP96345; SR48968; ezlopitant; CJ 11974; MEN11558; [18F] SPA-RQ; neuropitant 21; betupitant 29; SR 144190; SR48692; SR141716; L733060; vofopitant; R-673; nepadutant; saredutant; UK 290795; 2-(4-biphenylyl)quinoline-4-carboxylate and carboxamide analogs (neurokinin-3 receptor antagonist); 4-Amino-2-(aryl)-butylbenzamides and analogues; MK-869; L742694; CP 122721; 1-alkyl-5-(3,4-dichlorophenyl)-5-[2-[(3-substituted)-1-azetidinyl]ethyl]-2-piperidines; L760735; L758,298, Cbz-Gly-Leu-Trp-0Bzl(CF(3))(2); L733,061; SR144190; SB235375; N—[(R,R)-(E)-1-arylmethyl-3-(2-oxo-azepan-3-yl) carbamoyl]allyl-N-methyl-3,5-bis(trifluoromethyl)benzamides; 3-[N¹-3,5-bis (trifluoromethyl)benzoyl-N-arylmethyl-N¹-methylhydrazino]-N—[(R)-2-oxo-azepan-3-yl]propionanides; SR142806; SR48,968; CP141,938; LY306740; SB40023; SB414240; Nolpitantium; SR140333; perhydroisoindole RP 67580, Depitant; RPR 100893; Lanepitant; LY-303870; LY303870; sanoti synthelabo; nolpitanium; SR 140333; SR 48968; Savedutant; AV 608; AV-608, AV608; CGP 60829; NK-608; NKP-608C; NKP608; CS003; R113281; Vestipitant; 597599; GW 597599; GW 597599B; Nurokinin antagonist; SSR 240600; casopitant; 679769; GW 679769; TA 5538; SSR 146977; SLV317; SLV-317; 823296; GW 823296; AVE 5883; AVE-5883; AZ 311; SB 235375; SB 733210; AZ 685; SAR 102279; SAR 10279; SSR 241586; SLV 332; Neurokinin 2 antagonist-Solvay; NK-2 antagonist-Solvat; SLV-332; SLV332, NIK 616; MPV4505; NIK616; MPC 4505; Z501; Z-501; 1 TAK 637; CP 96345; L 659877; CGP 49823; GR 203040; L 732138; S 16474; WIN 51708; ZD 7944; S 18523; CI 1021; PD 154075; 758298; ZD 4974; S 18920; HMR 2091; FK 355; SCH 205528; NK 5807; NIP 531; SCH 62373; UK 224671; MEN 10627; WIN 64821; MDL 105212A; MEN 10573; TAC 363; 1 MEN 11149; HSP 117; NIP 530; and AZD 5106.

12. The method of claim 10, wherein the NK antagonist is aprepitant.

13. The method of claim 10 wherein the opioid is selected from morphine, fentanyl, oxycodone, codeine, dihydrocodeine, dihydrocodeinone enol acetate, desomorphine, apomorphine, diamorphine, pethidine, methadone, dextropropoxyphene, pentazocine, dextromoramide, oxymorphone, hydromorphone, dihydromorphine, papverine, papveretum, alfentanil, buprenorphine and tramadol and pharmaceutically acceptable derivates, homologs or analogs thereof.

14. The method of claim 10 wherein the pain is associated with a disease selected from Abdominal Wall Defect, Abdominal Migraine, Achondrogenesis, Achondrogenesis Type IV, Achondrogenesis Type III, Achondroplasia, Achondroplasia Tarda, Achondroplastic Dwarfism, Acquired Immunodeficiency Syndrome (AIDS), Acute Intermittent Porphyria, Acute Porphyrias, Acute Shoulder Neuritis, Acute Toxic Epidermolysis, Adiposa Dolorosa, Adrenal Neoplasm, Adrenomyeloneuropathy, Adult Dermatomyositis, Amyotrophic Lateral Sclerosis, Amyotrophic Lateral Sclerosis-Polyglucosan Bodies, AN, AN 1, AN 2, Anal Rectal Malformations, Anal Stenosis, Arachnitis, Arachnoiditis Ossificans, Arachnoiditis, Arteritis Giant Cell, Arthritis, Arthritis Urethritica, Ascending Paralysis, Astrocytoma Grade I (Benign), Astrocytoma Grade II (Benign), Athetoid Cerebral Palsy, Barrett Esophagus, Barrett Ulcer, Benign Tumors of the Central Nervous System, Bone Tumor-Epidermoid Cyst-Polyposis, Brachial Neuritis, Brachial Neuritis Syndrome, Brachial Plexus Neuritis, Brachial-Plexus-Neuropathy, Brachiocephalic Ischemia, Brain Tumors, Brain Tumors Benign, Brain Tumors Malignant, Brittle Bone Disease, Bullosa Hereditaria, Bullous CIE, Bullous Congenital Ichthyosiform Erythroderma, Bullous Ichthyosis, Bullous Pemphigoid, Burkitt's Lymphoma, Burkitt's Lymphoma African type, Burkitt's Lymphoma Non-African type, Calcaneal Valgus, Calcaneovalgus, Cavernous Lymphangioma, Cavernous Malformations, Central Form Neurofibromatosis, Cervical Spinal Stenosis, Cervical Vertebral Fusion, Charcot's Disease, Charcot-Marie-Tooth, Charcot-Marie-Tooth Disease, Charcot-Marie-Tooth Disease Variant, Charcot-Marie-Tooth-Roussy-Levy Disease, Childhood Dermatomyositis, Chondrodysplasia Punctata, Chondrodystrophia Calcificans Congenita, Chondrodystrophia Fetalis, Chondrodystrophic Myotonia, Chondrodystrophy, Chondrodystrophy with Clubfeet, Chondrodystrophy Epiphyseal, Chondrodystrophy Hyperplastic Form, Chondroectodermal Dysplasias, Chondrogenesis Imperfecta, Chondrohystrophia, Chondroosteodystrophy, Chronic Adhesive Arachnoiditis, Chronic Idiopathic Polyneuritis (CIP), Chronic Inflammatory Demyelinating Polyneuropathy, Chronic Inflammatory Demyelinating Polyradiculoneuropathy, Cicatricial Pemphigoid, Complex Regional Pain Syndrome, Congenital Cervical Synostosis, Congenital Dysmyelinating Neuropathy, Congenital Hypomyelinating Polyneuropathy, Congenital Hypomyelination Neuropathy, Congenital Hypomyelination, Congenital Hypomyelination (Onion Bulb) Polyneuropathy, Congenital Ichthyosiform Erythroderma, Congenital Tethered Cervical Spinal Cord Syndrome, Cranial Arteritis, Crohn's Disease, Cutaneous Porphyrias, Degenerative Lumbar Spinal Stenosis, Demyelinating Disease, Diabetes Mellitus Diabetes Insulin Dependent, Diabetes Mellitus, Diabetes Mellitus Addison's Disease Myxedema, Discoid Lupus, Discoid Lupus Erythematosus, Disseminated Lupus Erythematosus, Disseminated Neurodermatitis, Disseminated Sclerosis, EDS Kyphoscoliotic, EDS Kyphoscoliosis, EDS Mitis Type, EDS Ocular-Scoliotic, Elastosis Dystrophica Syndrome, Encephalofacial Angiomatosis, Encephalotrigeminal Angiomatosis, Enchondromatosis with Multiple Cavernous Hemangiomas, Endemic Polyneuritis, Endometriosis, Eosinophilic Fasciitis, Epidermolysis Bullosa, Epidermolysis Bullosa Acquisita, Epidermolysis Bullosa Hereditaria, Epidermolysis Bullosa Letalias, Epidermolysis Hereditaria Tarda, Epidermolytic Hyperkeratosis, Epidermolytic Hyperkeratosis (Bullous CIE), Familial Lumbar Stenosis, Familial Lymphedema Praecox, Fibromyositis, Fibrositis, Fibrous Ankylosis of Multiple Joints, Fibrous Dysplasia, Fragile X syndrome, Generalized Fibromatosis, Guillain-Barre Syndrome, Hemangiomatosis Chondrodystrophica, Hereditary Sensory and Autonomic Neuropathy Type I, Hereditary Sensory and Autonomic Neuropathy Type II, Hereditary Sensory and Autonomic Neuropathy Type III, Hereditary Sensory Motor Neuropathy, Hereditary Sensory Neuropathy type I, Hereditary Sensory Neuropathy Type I, Hereditary Sensory Neuropathy Type II, Hereditary Sensory Neuropathy Type III, Hereditary Sensory Radicular Neuropathy Type I, Hereditary Sensory Radicular Neuropathy Type I, Hereditary Sensory Radicular Neuropathy Type II, Herpes Zoster, Hodgkin Disease, Hodgkin's Disease, Hodgkin's Lymphoma, Hyperplastic Epidermolysis Bullosa, Hypertrophic Interstitial Neuropathy, Hypertrophic Interstitial Neuritis, Hypertrophic Interstitial Radiculoneuropathy, Hypertrophic Neuropathy of Refsum, Idiopathic Brachial Plexus Neuropathy, Idiopathic Cervical Dystonia, Juvenile (Childhood) Dermatomyositis (JDMS), Juvenile Diabetes, Juvenile Rheumatoid Arthritis, Pes Planus, Leg Ulcer, Lumbar Canal Stenosis, Lumbar Spinal Stenosis, Lumbosacral Spinal Stenosis, Lupus, Lupus, Lupus Erythematosus, Lymphangiomas, Mononeuritis Multiplex, Mononeuritis Peripheral, Mononeuropathy Peripheral, Monostotic Fibrous Dysplasia, Multiple Cartilaginous Enchondroses, Multiple Cartilaginous Exostoses, Multiple Enchondromatosis, Multiple Myeloma, Multiple Neuritis of the Shoulder Girdle, Multiple Osteochondromatosis, Multiple Peripheral Neuritis, Multiple Sclerosis, Musculoskeletal Pain Syndrome, Neuropathic Amyloidosis, Neuropathic Beriberi, Neuropathy of Brachialpelxus Syndrome, Neuropathy Hereditary Sensory Type I, Neuropathy Hereditary Sensory Type II, Nieman Pick disease Type A (acute neuronopathic form), Nieman Pick disease Type B, Nieman Pick Disease Type C (chronic neuronopathic form), Non-Scarring Epidermolysis Bullosa, Ochronotic Arthritis, Ocular Herpes, Onion-Bulb Neuropathy, Osteogenesis Imperfect, Osteogenesis Imperfecta, Osteogenesis Imperfecta Congenita, Osteogenesis Imperfecta Tarda, Peripheral Neuritis, Peripheral Neuropathy, Perthes Disease, Polyarteritis Nodosa, Polymyalgia Rheumatica, Polymyositis and Dermatomyositis, Polyneuritis Peripheral, Polyneuropathy Peripheral, Polyneuropathy and Polyradiculoneuropathy, Polyostotic Fibrous Dysplasia, Polyostotic Sclerosing Histiocytosis, Postmyelographic Arachnoiditis, Primary Progressive Multiple Sclerosis, Psoriasis, Radial Nerve Palsy, Radicular Neuropathy Sensory, Radicular Neuropathy Sensory Recessive, Reflex Sympathetic Dystrophy Syndrome, Relapsing-Remitting Multiple Sclerosis, Sensory Neuropathy Hereditary Type I, Sensory Neuropathy Hereditary Type II, Sensory Neuropathy Hereditary Type I, Sensory Radicular Neuropathy, Sensory Radicular Neuropathy Recessive, Sickle Cell Anemia, Sickle Cell Disease, Sickle Cell-Hemoglobin C Disease, Sickle Cell-Hemoglobin D Disease, Sickle Cell-Thalassemia Disease, Sickle Cell Trait, Spina Bifida, Spina Bifida Aperta, Spinal Arachnoiditis, Spinal Arteriovenous Malformation, Spinal Ossifying Arachnoiditis, Spinal Stenosis, Stenosis of the Lumbar Vertebral Canal, Still's Disease, Syringomyelia, Systemic Sclerosis, Talipes Calcaneus, Talipes Equinovarus, Talipes Equinus, Talipes Varus, Talipes Valgus, Tandem Spinal Stenosis, Temporal Arteritis/Giant Cell Arteritis, Temporal Arteritis, Tethered Spinal Cord Syndrome, Tethered Cord Malformation Sequence, Tethered Cord Syndrome, Tethered Cervical Spinal Cord Syndrome, Thalamic Pain Syndrome, Thalamic Hyperesthetic Anesthesia, Trigeminal Neuralgia, Variegate Porphyria and Vertebral Ankylosing Hyperostosis.

15. Use of CNSB002 in the manufacture of a medicament to ameliorate the symptoms of inflammatory or neuropathic pain.

16. Use of claim 15 wherein the medicament further comprises an opioid and/or an NK antagonist.

17. A system for the controlled release of CNSB002 or CNSB002, an opioid and/or an NK antagonist:
(a) a deposit-core comprising an effective amount of a first active substance and having defined geometric form, and
(b) a support-platform applied to the deposit-core, wherein the support-platform contains a second active substance, and at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.
